SEQUENCE LISTING (6 sequences)

SEQ ID NO: 1 - RNA, 23 nt, Homo sapiens
uuaaugcuaaucgugauaggggu

SEQ ID NO: 2 - RNA, 23 nt, Mus musculus
uuaaugcuaauugugauaggggu

SEQ ID NO: 3 - RNA, 65 nt, Homo sapiens
cuguuaaugcuaaucgugauagggguuuuugccuccaacugacuccuacauauuagcauuaacag

SEQ ID NO: 4 - RNA, 65 nt, Mus musculus
cuguuaaugcuaauugugauagggguuuuggccucugacugacuccuaccuguuagcauuaacag

SEQ ID NO: 5 - protein, 8 aa, Artificial Sequence
  Synthetic
SIINFEKL

SEQ ID NO: 6 - protein, 8 aa, Artificial Sequence
  Synthetic
SIITFEKL

(19) United States
(12) Patent Application Publication
(10) Pub. No.: US 2015/0275209 A1
(43) Pub. Date: Oct. 1, 2015

(54) COMPOSITIONS AND METHODS FOR ENHANCING CANCER IMMUNOTHERAPY

(71) Applicant: THE UNITED STATES OF AMERICA, AS REPRESENTED BY THE SECRETARY DEPT, OF HEALTH AND HUMAN SERVICES, Bethesda, MD (US)

(72) Inventors: Yun Ji, Boyds, MD (US); Nicholas P. Restifo, Chevy Chase, MD (US); Luca Gattinoni, Washington, DC (US)

(73) Assignee: The United States of America, as represented by the Secretary, Dept. of Health and Human Service, Bethesda, MD (US)

(21) Appl. No.: 14/436,947

(22) PCT Filed: Oct. 17, 2013
(86) PCT No.: PCT/US2013/065452

§ 371 Date: Apr. 20, 2015

Related U.S. Application Data
(60) Provisional application No. 61/716,653, filed on Oct. 22, 2012.

(51) Int. Cl. C12N 15/113; A61K 39/00; A61K 35/17

(52) U.S. Cl. CPC C12N 15/113; A61K 35/17; A61K 39/0011; C12N 2310/141; C12N 2320/30

(57) ABSTRACT

The invention provides an isolated or purified CD8+? T cell which comprises an antigen-specific T cell receptor and an exogenous nucleic acid encoding a microRNA-155 (miR-155) molecule, and methods of preparing the same. The invention also provides a pharmaceutical composition comprising the CD8+ T cell a carrier. Further provided is a method for treating or preventing a medical condition, such as cancer, by adoptively transferring to a mammal an amount of the CD8+? T cells effective to treat or prevent the medical condition.

### CROSS-REFERENCE TO RELATED APPLICATIONS

This application claims the benefit of U.S. Provisional Patent Application 61/716,653, filed Oct. 22, 2012, which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,432 Byte ASCII (Text) file named “714365ST25.TXT,” created on Oct. 16, 2013.

### BACKGROUND OF THE INVENTION

Adoptive cell transfer (ACT) refers to the treatment of a patient with a cell population which has been expanded ex vivo. Immunotherapy based upon the adoptive transfer of naturally occurring tumor infiltrating lymphocyte (TIL) populations has been demonstrated to mediate tumor regression in cancer patients, particularly in cancer patients with metastatic melanoma. The cellular mechanisms that mediate antitumor responses following ACT are complex and involve both CD8⁺ T cells and CD4⁺ T cells, among other cell types. In order to enhance the efficacy of an ACT-based therapy, T cells can be genetically engineered ex vivo prior to infusion into a cancer patient. For example, T cells can be genetically engineered to express a T cell receptor (TCR) with a high affinity and specificity for a particular tumor antigen, thereby increasing the range of tumor types susceptible to treatment. In addition, T cells can be genetically engineered to express one or more molecules which enhance co-stimulation, prevent apoptosis, induce inflammation, promote homeostatic proliferation, and/or enhance T cell homing (Restifo et al., Nat. Rev. Immunol., 12: 269-281 (2012)).

There is evidence that microRNAs (miRNAs) are involved in immune system function. For example, bic-deficient mice, which do not produce microRNA-155 (miR-155; also referred to as Mir155), are immunodeficient, displaying impaired B cell responses and an intrinsic bias of CD4⁺ T cells towards Th2 differentiation (Rodriguez et al., Science, 316: 608-611 (2007)). miR-155-deficient (Mir155^{-/-}) mice are resistant to experimental autoimmune encephalomyelitis (O'Connell et al., Immunity, 33: 607-619 (2010)), and to experimental acute graft-versus-host disease (Ranganathan et al., Blood, 119 (20): 4786-4797 (2012)). It also has been demonstrated that the expression of miR-155 is increased in activated CD4⁺ T cells (O'Connell et al. supra), and during CD8⁺ T cell differentiation (Salaun et al., J. Translational Medicine, 9: 44 (2011)).

There is a need in the art for improved compositions and methods for immunotherapy based upon the adoptive transfer of T cells. This invention provides such compositions and methods, which can be useful for the treatment of cancer.

### BRIEF SUMMARY OF THE INVENTION

The invention provides an isolated or purified CD8⁺ T cell which comprises (a) an antigen-specific T cell receptor (TCR) and (b) an exogenous nucleic acid encoding a microRNA-155 (miR-155) molecule.

The invention also provides a population of cells comprising at least one of the CD8⁺ T cells comprising an antigen-specific TCR and an exogenous miR-155, as well as a composition comprising the population of cells and a carrier.

The invention further provides a method of treating cancer by administering to a cancer patient the population of cells comprising at least one of the CD8⁺ T cells comprising an antigen-specific TCR and an exogenous miR-155, or a composition thereof.

### BRIEF DESCRIPTION OF THE SEVERAL VIEWS OF THE DRAWING(S)

FIG. 1A is a fluorescence-activated cell sorting (FACS) plot depicting the expression of green fluorescent protein (GFP) in CD8⁺ T cells expressing miR-155 or a scrambled version of miR-155. FIG. 1B is a bar graph depicting the relative expression of miR-155 as compared to the expression of U6 small nuclear RNA (snRNA) in CD8⁺ T cells expressing miR-155 or a scrambled version of miR-155, as assessed by quantitative PCR (qPCR). **** p<0.0001.

FIG. 2 includes FACS plots depicting production of CD44, CD62L, IL7rα, and IL7rβ in CD8⁺ T cells expressing miR-155 or a scrambled version of miR-155.

FIGS. 3A and 3B are line graphs depicting tumor size at various time points in untreated mice (NT), or in mice adoptively transferred with CD8⁺ T cells overexpressing miR-155 or scrambled miR-155. The data depicted in FIG. 3A were obtained with gp100-vaccinated mice, whereas the data depicted in FIG. 3B were obtained with unvaccinated mice.

FIG. 4 is a line graph depicting tumor size at various time points in untreated mice (NT), or in gp-100 vaccinated mice adoptively transferred with CD8⁺ T cells overexpressing miR-155 or scrambled miR-155 which also received a dose of exogenous IL-2 at 0.5, 12, 24, and 36 hours following ACT.

FIGS. 5A and 5B are line graphs depicting tumor size at various time points in untreated mice (NT), or in gp-100 vaccinated mice adoptively transferred with CD8⁺ T cells overexpressing miR-155 or scrambled miR-155, without irradiation prior to ACT (FIG. 5A) or with 6 Gy irradiation prior to ACT (FIG. 5B).

FIGS. 6A and 6B includes line graphs depicting tumor size (FIG. 6A) or overall survival (FIG. 6B) at various time points without ACT (NT), or following ACT of CD8⁺ T cells overexpressing miR-155 or scrambled miR-155 in wild-type (WT), CD4-deficient (CD4^{-/-}), CD8-deficient (CD8^{-/-}), or RAG-1-deficient (RAG^{-/-}) mice. * p<0.05; ** p<0.01.

FIG. 7 is a plot depicting the percentage of CD8 and GFP double positive cells per spleen at the indicated number of days (d) following ACT of CD8⁺ T cells overexpressing miR-155 or scrambled miR-155 in WT mice. *p<0.05; **p<0.01; ***p<0.001.

FIGS. 8A and 8B include FACS plots depicting IFN-γ, IL-2, and TNF-α production in splenocytes obtained from mice at day 4 or 6 following adoptive transfer of CD8⁺ T cells expressing miR-155 or a scrambled version of miR-155.

FIG. 9 is a graph which depicts experimental data illustrating the number of cytokine releasing CD8 and GFP double positive cells per spleen at the indicated number of days following ACT of CD8⁺ T cells overexpressing miR-155 (155) or scrambled miR-155 (s) in wild-type (WT) mice or Il-7^{-/-}Il-15^{-/-} (DKO) mice infected with rvvhgp100.

FIG. 10A is a graph which depicts experimental data illustrating quantification of miR-155 in human CD8+ T cells transduced with TCRs of increasing affinity following TCR stimulation with multimers (N=3 experiments) as fold increase relative to unstimulated clones. FIG. 10B is a graph which depicts experimental data illustrating miR-155 expression of naïve mouse OT-1 T cells stimulated with splenic dendritic cells pulsed with the natural SIITFEKL (N4) peptide (SEQ ID NO: 5) or weaker SIITFEKL (T4) altered peptide ligand (SEQ ID NO: 6) as relative to day 0 unstimulated cells. Data are representative for triplicates in one out of two experiments. FIG. 10C is a graph which depicts experimental data illustrating miR-155 concentrations in naïve, central memory, and effector CD8⁺ T cells sorted at day 8 after LCMV strain WE infection as fold change relative to naïve cells. FIG. 10D is a graph which depicts experimental data illustrating relative miR-155 expression in splenic naïve and effector CD8⁺ T cells sorted from LCMV infected mice. Symbols represent individual mice, and the line is the mean +/− SEM.

FIG. 11A is a graph which depicts experimental data illustrating expression of LCMV gp33 tetramers and CD8 in splenocytes from LCMV-infected wild-type (WT) and Mir155^{-/-} mice at 8 days post-infection. FIG. 11B are graphs which depict experimental data illustrating blood percentages and numbers of CD8⁺ (upper panels) and gp33 tetramer⁺ cells (lower panels) on day 8 post-infection. FIG. 11C are graphs which depict experimental data illustrating percentages of CD44^{high}CD62L^{low} effector CD8⁺ T cells gated on lymphocytes in wild-type (WT) and Mir155^{-/-} spleen cells (upper panel) and of gp33 tetramer⁺ cells within the CD8⁺ T cells (lower panel) at days 6 to 8 post LCMV-infection. FIG. 11D are graphs which depict percentages of total CD8⁺ T cells (left panel) and CD127⁺ cells within tetramer gp33⁺ CD8⁺ cells (right panel) in blood at the indicated time points. FIGS. 11E and 11F are graphs which depict experimental data illustrating the percentage of liver CD127^{high}CD62L^{high} tetramer gp33⁺ and np396⁺ memory cells (FIG. 11E) and IL-2 production upon gp33 peptide restimulation of splenocytes within IFN-γ positive CD8⁺ T cells (FIG. 11F) at 3 months past infection. Symbols represent individual mice, and the line is the mean+/−SEM.

FIGS. 12A-F include graphs which depict experimental data illustrating that miR-155 promotes effector CD8⁺T cells, as described in Example 9. FIG. 12A includes graphs which depict experimental data illustrating the ratio of congenically marked wild-type (WT):Mir155^{-/-} OT-1 cells competitively cocultured with peptide-pulsed dendritic cells at the indicated time points (left panel). On day 5, the percentage of trypan blue cells harvested from WT or Mir155^{-/-} cultures was counted (right panel). Pooled data from three representative experiments are pictured. FIG. 12B is a graph which depicts experimental data illustrating the percentage of CD8⁺ T cells from WT and Mir155^{-/-} mice cotransferred into WT or deficient hosts eight days post-infection with LCMV strain WE. FIG. 12C is a graph which depicts experimental data illustrating CD8⁺ effector T cell ratios isolated from Rag2 and IL2Rγ double deficient mice into which a 1:1 mix of WT and Mir155^{-/-} splenocytes was adoptively transferred. Mice were infected with LCMV WE strain two months after transfer, and data at days 1 and 8 post-infection are pictured. FIG. 12D is a graph which depicts experimental data illustrating proliferating BrdU positive splenic CD44^{high}CD8⁺ effector T cells at days 6 and 7 post LCMV infection. FIG. 12E is a graph which depicts experimental data illustrating expression of the proliferation marker Ki67 in splenic CD44^{high} CD8⁺ effector T cells at day 7 post LCMV infection. FIG. 12F is a graph which depicts experimental data illustrating identification of apoptotic cells by AnnexinV staining. Symbols represent individual mice, and the line is the mean+/−SEM.

FIGS. 13A-F include graphs which depict experimental data illustrating that miR-155 induces survival of effector cells and sustains the anti-viral response in a chronic LCMV infection, as described in Example 10. Effector CD44^{high}CD62L^{low} within blood CD8⁺ T cells are shown in FIG. 13A at the indicated time points. FIGS. 13B and 13C are graphs which illustrate expression of activation markers and gp33 tetramer⁺ cells (FIG. 13B) and flow cytometry dot blots (FIG. 13C) from representative mice. FIGS. 13D and 13E are graphs which illustrate virus titer (FIG. 13D) and cytokine response (FIG. 13E) upon stimulation with a peptide cocktail in the blood at two and three months after LCMV infection, respectively. FIG. 13F is a graph which illustrates the weight of mice during the first two weeks post-infection. Symbols represent individual mice, and the line is the mean. Error bars are given as +/−SEM.

FIG. 14A is a graph which depicts experimental data illustrating the ratio of antigen-specific versus polyclonal cells in the draining lymph nodes of WT mice adoptively transferred with congenically marked OT-1 and polyclonal CD8⁺ T cells from wild-type (WT) or Mir155^{-/-} backgrounds four days after immunization with OVA peptide and CpG in IFA. FIG. 14B is a graph which depicts experimental data illustrating the ratio of blood WT and Mir155^{-/-} OT-1 CD8⁺ T cells cotransferred into WT hosts, which were immunized as in FIG. 14A on day 7. Symbols represent individual mice, and the line is the mean +/− SEM.

FIGS. 15A-E include graphs which depict experimental data illustrating that SOCS-1 and miR-155 modulate the antiviral CD8⁺ T cell response and cytokine signaling, as described in Example 11. FIG. 15A illustrates SOCS-1 mRNA concentrations measured upon LCMV WE infection in purified effector (CD44^{high}CD62L^{low}), wild-type (WT), and Mir155^{-/-} splenic CD8⁺ T cells by qPCR relative to naïve CD8⁺ T cells from non-infected mice. FIGS. 15B and 15C illustrate regulation of SOCS-1 expression by miR-155 in naïve CD8⁺ T cells from WT and miR-155 deleted mice as well as after retroviral transfection with miR-155 overexpressing or control vectors tested by qPCR (shown as relative to β-actin) (FIG. 15B) and immunoblot (FIG. 15C). FIG. 15D is a graph illustrating pSTAT5 expression in naïve or effector T cells from LCMV infected mice stimulated with the indicated cytokines as measured by flow cytometry. FIG. 15E includes graphs illustrating the pSTAT5 response to IL-2 in WT and Mir155^{-/-} T cells transduced with control or shSOCS-1 lentivirus. The right panel gives the percentages of mean fluorescence intensity (MFI) normalized to the MFI measured in wild-type sh-control cells set to 100%.

FIGS. 16A-D include graphs which depict experimental data illustrating that SOCS-1 limits the CD8⁺ T cell response to virus and cancer, as described in Example 12. TCR transgenic P14 CD8⁺ T cells with or without SOCS-1 (P14×SOCS-1) overexpression were adoptively transferred before LCMV WE infection. Data in FIG. 16A show the percentage of transferred cells in the lymphocyte gate at days 6, 7, and 8 post-infection as mean+/−SEM. FIG. 16B illustrates apoptotic cells within P14 T cells 7 days post-infection. Symbols represent single mice and the line is the mean. TCR transgenic pmel-1 CD8⁺ T cells were transduced with a retrovirus encoding a scrambled control or shSOCS-1 mRNA and adoptively transferred into tumor bearing mice. FIG. 16C illustrates the absolute numbers of donor CD8⁺ T cells determined in spleen at days 4 to 6 after adoptive transfer, and FIG. 16D shows the change in tumor size of mice after adoptive transfer. Data are from one representative out of two independent experiments with two (FIG. 16C) to five mice (FIG. 16D) per group and displayed as mean+/−SEM.

FIGS. 17A-17E are images of immunoblots of pmel-1 CD8⁺ T cells transduced with miR-155 or scramble miR, probed with anti-pMAPK (FIG. 17A), anti-Ptpn2 (FIG. 17B), anti-SOCS1 (FIG. 17C), anti-SHIP1 (FIG. 17D) and anti-p-Akt (FIG. 17E), as described in Example 13.

FIGS. 18A and 18B are graphs which depict experimental data illustrating the quantification of GFP⁺Thy1.1⁺ pmel-1 CD8⁺ T cells after adoptive transfer of cells as described in Example 12. FIGS. 18C and 18D are graphs which depict experimental data illustrating the relative ratio of miR-155 overexpressing cells compared to scramble miR in the presence of constitutive Stat5a (FIG. 18C), Akt (FIG. 18D), or control Thy1.1.

### DETAILED DESCRIPTION OF THE INVENTION

The invention provides an isolated or purified CD8⁺ T cell, or a population thereof, as well as compositions, e.g., pharmaceutical compositions, comprising the same, and methods of preparing the same. The isolated or purified CD8⁺ T cell of the invention comprises at least two elements, namely an antigen-specific TCR and an exogenous nucleic acid encoding a miR-155 molecule.

The term “isolated” as used herein means having been removed from its natural environment. The term “purified” as used herein means having been increased in purity, wherein “purity” is a relative term, and not to be necessarily construed as absolute purity. A “purified” CD8⁺ T cell refers to a CD8⁺ T cell which has been separated from other natural components, such as tissues, cells, proteins, nucleic acids, etc.

The inventive compositions can comprise a single CD8⁺ T cell or a population thereof. The population of CD8⁺ T cells can be a heterogeneous population comprising the CD8⁺ T cell expressing an exogenous miR-155, in addition to at least one other cell, e.g., a CD8⁺ T cell, which does not express an exogenous miR-155, or a cell other than a CD8⁺ T cell, e.g., a CD4⁺ T cell, a B cell, a macrophage, a neutrophil, an erythrocyte, a melanocyte, a hepatocyte, an endothelial cell, an epithelial cell, a muscle cell, a brain cell, etc. Alternatively, the population of CD8⁺ T cells can be a substantially homogeneous population, in which the population mainly comprises CD8⁺ T cells expressing an exogenous miR-155. The population also can be a clonal population of CD8⁺ T cells, in which all CD8⁺ T cells of the population are clones of a single CD8⁺ T cell expressing an exogenous nucleic acid encoding miR-155, such that all CD8⁺ T cells of the population express miR-155 and have genetically identical TCRs.

A CD8⁺ T cell of the invention can be present in a population of cells or a composition in an amount of 10% or more, e.g., 30% or more, 50% or more, 60% or more, 70% or more, 75% or more, 80% or more, 85% or more, or 90% or more, based on the total number of cells in the population or composition. Alternatively, or in addition, the CD8⁺ T cell of the invention can be present in a population of cells or a composition in an amount of 95% or less, e.g., 90% or less, 85% or less, 80% or less, 75% or less, 70% or less, 60% or less, 40% or less, or 30% or less based on the total number of cells in the population or composition. Thus, the CD8⁺ T cell of the invention can be present in a population of cells or a composition in an amount bounded by any two of the above endpoints. For example, the CD8⁺ T cell of the invention can be present in a population of cells or a composition in an amount of 30-60%, 10-40%, 50-90%, 60-80%, 30-95%, 80-90%, or 75-85%.

The CD8⁺ T cell can be any T cell which displays cell surface expression of the CD8 glycoprotein. The CD8⁺ T cell can be a cultured CD8⁺ T cell, e.g., a primary CD8⁺ T cell, or a CD8⁺ T cell from a cultured CD8⁺ T cell line, e.g., Jurkat, SupT1, etc., or a CD8⁺ T cell obtained from a mammal, e.g., a human. If obtained from a human or other mammal, the CD8⁺ T cell can be isolated from numerous sources, including but not limited to blood, bone marrow, lymph node, thymus, spleen, or other tissues or fluids.

The CD8⁺ T cell, or populations thereof, as well as the compositions comprising the same, have many uses. Preferred uses include the treatment or prevention of a medical condition, e.g., a disease such as cancer, infectious disease, and autoimmune disease, or immunodeficiency. In this respect, the CD8⁺ T cell of the invention can comprise a TCR specific for an antigen of a medical condition. The TCR can be an antigen-specific receptor which recognizes any antigen that is characteristic of the medical condition, e.g., disease, to be treated or prevented, as discussed herein.

By “antigen-specific TCR” is meant a TCR which can specifically bind to and immunologically recognize an antigen, or an epitope thereof, such that binding of the TCR to antigen, or the epitope thereof, elicits an immune response. The antigen-specific TCR generally comprises two polypeptides (i.e., polypeptide chains), such as an α-chain of a TCR, a β-chain of a TCR, a γ-chain of a TCR, a δ-chain of a TCR, or a combination thereof. Such polypeptide chains of TCRs are known in the art. The antigen-specific TCR can comprise any amino acid sequence, provided that the TCR can specifically bind to and immunologically recognize an antigen, such as a medical condition- or disease-associated antigen or epitope thereof.

The antigen-specific TCR can be an endogenous TCR, i.e., the antigen-specific TCR that is endogenous or native to (naturally-occurring on) the CD8⁺ T cell. In such a case, the CD8⁺ T cell comprising the endogenous TCR can be a CD8⁺ T cell that was isolated from a mammal which is known to express the particular medical condition-specific antigen. In certain embodiments, the CD8⁺ T cell is a primary CD8⁺ T cell isolated from a host afflicted with cancer. In some embodiments, the CD8⁺ T cell is a tumor infiltrating lymphocyte (TIL) or a peripheral blood lymphocyte (PBL) isolated from a human cancer patient.

In some embodiments, the mammal from which a CD8⁺ T cell is isolated is immunized with an antigen of, or specific for, a medical condition, e.g., a disease. Desirably, the mammal is immunized prior to obtaining the CD8⁺ T cell from the mammal. In this way, the isolated CD8⁺ T cells can include CD8⁺ T cells induced to have specificity for the medical condition to be treated, or can include a higher proportion of cells specific for the medical condition.

Alternatively, a CD8⁺ T cell comprising an endogenous antigen-specific TCR can be a CD8⁺ T cell within a mixed population of cells isolated from a mammal, and the mixed population can be exposed to the antigen which is recognized by the endogenous TCR while being cultured in vitro. In this manner, the CD8⁺ T cell which comprises the TCR that recognizes the medical condition-specific antigen, expands or proliferates in vitro, thereby increasing the number of T lymphocytes having the endogenous antigen-specific receptor.

The antigen-specific TCR also can be a recombinant TCR, e.g., a TCR which has been generated through recombinant expression of one or more exogenous TCR α-, β-, γ-, and/or δ-chain encoding genes. A recombinant TCR can comprise polypeptide chains derived entirely from a single mammalian species, or the antigen-specific TCR can be a chimeric or hybrid TCR comprised of amino acid sequences derived from TCRs from two different mammalian species. For example, the antigen-specific TCR can comprise a variable region derived from a murine TCR, and a constant region of a human TCR such that the TCR is “humanized.” Methods of making such hybrid TCRs are known in the art. See, for example, Cohen et al., Cancer Res. 66: 8878-8886 (2006).

A CD8⁺ T cell of the invention comprising an endogenous antigen-specific TCR can also be transformed, e.g., transduced or transfected, with one or more nucleic acids encoding an exogenous (e.g., recombinant) TCR or other recombinant chimeric receptor. Such exogenous chimeric receptors, e.g., chimeric TCRs, can confer specificity for additional antigens to the transformed CD8⁺ T cell beyond the antigens for which the endogenous TCR is naturally specific. This can, but need not, result in the production of CD8⁺ T cell having dual antigen specificities.

Chimeric TCRs also are referred to in the art as “chimeric antigen receptors” (CARs). Typically, a CAR comprises the antigen binding domain of an antibody, e.g., a single-chain variable fragment (scFv), fused to the transmembrane and intracellular domains of a TCR. Thus, the antigenic specificity of a TCR of the invention can be encoded by a scFv which specifically binds to the antigen, or an epitope thereof. Methods of making such chimeric TCRs are known in the art. See, for example, U.S. Patent Application Publication 2012/0213783.

Any suitable nucleic acid encoding a chimeric receptor, TCR, or TCR-like protein can be used. TCRs for use in this embodiment are known in the art. For example, polynucleotides encoding TCRs for gp100, NY-ESO-1, and MART-1 have been used in immunotherapy. See, for example, U.S. Pat. No. 5,830,755; Zhao et al., J. Immunology 174 (7): 4415-23 (2005); and Hughes et al., Hum Gene Ther. 16 (4): 457-472 (2005). In these embodiments, transformation with a nucleic acid encoding a miR-155 molecule as discussed below, can occur before, after, or simultaneously with, TCR transformation. The TCR encoded by the transformed nucleic acids can be of any suitable form including for example, a single-chain TCR or a fusion with other proteins (e.g., without limitation co-stimulatory molecules).

The antigen which is recognized by the antigen-specific TCR can be any antigen which is characteristic of a disease or a medical condition. For example, the antigen may be, but is not limited to, a tumor antigen (also termed tumor associated antigen) or a viral antigen. Tumor antigens are known in the art and include, for instance, gp100, MART-1, TRP-1, TRP-2, tyrosinase, NY-ESO-1 (also known as CAG-3), MAGE-1, MAGE-3, etc. Viral antigens are also known in the art and include, for example, any viral protein, e.g., env, gag, pol, gp120, thymidine kinase, and the like.

The disease or medical condition which is associated with or is characterized by the antigen recognized by the antigen-specific TCR can be any disease or medical condition. For instance, the disease or medical condition can be a cancer, an infectious disease, an autoimmune disease, or an immunodeficiency, as discussed herein.

In certain embodiments, the antigen-specific TCR of the invention preferably has specificity for a cancer antigen. The antigen-specific TCR can have specificity for an antigen derived from any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer; pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In certain preferred embodiments, the antigen-specific TCR has specificity for an antigen derived from colorectal cancer or melanoma.

For purposes herein, “infectious disease” means a disease that can be transmitted from person to person or from organism to organism, and is caused by a microbial agent (e.g., common cold). Infectious diseases are known in the art and include, for example, hepatitis, sexually transmitted diseases (e.g., Chlamydia, gonorrhea), tuberculosis, HIV/AIDS, diphtheria, hepatitis B, hepatitis C, cholera, and influenza.

For purposes herein, “autoimmune disease” refers to a disease in which the body produces an immunogenic (i.e., immune system) response to some constituent of its own tissue. In other words the immune system loses its ability to recognize some tissue or system within the body as “self” and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

For purposes herein, “immunodeficiency” means the state of a patient whose immune system has been compromised by disease or by administration of chemicals. This condition makes the system deficient in the number and type of blood cells needed to defend against a foreign substance. Immunodeficiency conditions or diseases are known in the art and include, for example, AIDS (acquired immunodeficiency syndrome), SCID (severe combined immunodeficiency disease), selective IgA deficiency, common variable immunodeficiency, X-linked agammaglobulinemia, chronic granulomatous disease, hyper-IgM syndrome, and diabetes.

A CD8⁺ T cell comprising an antigen-specific TCR can be isolated or purified from a source using any suitable technique known in the art. For example, a CD8⁺ T cell comprising an antigen-specific TCR present in a mammalian tissue, biological fluid (e.g., blood), or in vitro culture medium can be separated from impurities, e.g., other cell types, proteins, nucleic acids, etc. using flow cytometry, immunomagnetic separation, or a combination thereof.

A CD8⁺ T cell so obtained is then contacted, e.g., transduced or transfected, with an exogenous nucleic acid encoding a miR-155 molecule. Preferably, the exogenous nucleic acid is a recombinant nucleic acid. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, double- and single-stranded RNA, and double-stranded DNA-RNA hybrids. The terms include, as equivalents, analogs of either RNA or DNA made from nucleotide analogs and modified polynucleotides such as, though not limited to methylated and/or capped polynucleotides. Suitable nucleotide analogs are known and are described in, e.g., U.S. Pat. No. 6,107,094, U.S. Patent Application Publication 2012/0101148, and references cited therein.

The term “nucleotide” as used herein refers to a monomeric subunit of a polynucleotide that consists of a heterocyclic base, a sugar, and one or more phosphate groups. The naturally occurring bases (guanine (G), adenine (A), cytosine (C), thymine (T), and uracil (U)) are typically derivatives of purine or pyrimidine, though the invention includes the use of naturally and non-naturally occurring base analogs. The naturally occurring sugar is the pentose (five-carbon sugar) deoxyribose (which forms DNA) or ribose (which forms RNA), though the invention includes the use of naturally and non-naturally occurring sugar analogs. Nucleic acids are typically linked via phosphate bonds to form nucleic acids or polynucleotides, though many other linkages are known in the art (e.g., phosphotothioates, boranophosphates, and the like). Methods of preparing polynucleotides are within the ordinary skill in the art (Sambrook and Russell, Molecular Cloning: A Laboratory Manual, Cold Spring Harbor Laboratory Press, New York (2001)).

MiRNAs are non-coding small RNA molecules of generally about 19-25 nucleotides in length present in the genomes of a wide range of plants and animals. Mature miRNAs are processed from pre-miRNAs having a hairpin loop structure, which, in turn, are processed from primary RNA transcripts, often referred to as pri-miRNAs, pri-mirs or pri-pre-miRNAs. The 19-25 nucleotide miRNA molecule is also referred to in the art and herein as a “processed” miRNA gene transcript or “mature” miRNA.

Generally, a microRNA is incorporated into a microRNA-ribonucleoprotein complex, which mediates repression of a target mRNA sequence through a mechanism which involves mRNA cleavage or translational repression. It is believed that a miRNA will direct mRNA cleavage if the miRNA has sufficient complementarity to a target mRNA (e.g., 100% complementarity), or the miRNA will repress translation if the miRNA does not have sufficient complementarity to a target mRNA to mediate mRNA cleavage but does have a suitable amount of mRNA complementary sites. Thus, a miRNA does not need to be 100% complementary to a target mRNA sequence to repress target function (see, e.g., Bartel, Cell, 116: 281-297 (2004)).

An exogenous nucleic acid encoding a miR-155 molecule can consist of the nucleic acid sequence of the miR-155 molecule, i.e., wherein no additional nucleic acid sequences are present on the exogenous nucleic acid. An exogenous nucleic acid encoding a miR-155 molecule also can consist essentially of the nucleic acid sequence of a miR-155 molecule, i.e., wherein additional nucleic acid sequences may be present on the exogenous nucleic acid but the additional nucleic acid sequences do not substantially affect miR-155 expression, stability, or function. However, it is preferred that an exogenous nucleic acid encoding a miR-155 molecule comprises the nucleic acid sequence encoding the miR-155 molecule and additional nucleic acid sequences which regulate expression of the miR-155 molecule. Generally, an exogenous nucleic acid encoding a miR-155 molecule is carried in a plasmid or a viral vector which contains one or more regulatory nucleic acid sequences which provide for the miR-155 expression.

The miR-155 molecule of the invention can have any length provided that the miR-155 molecule retains the ability to base pair with one or more target mRNAs and repress target function. The miR-155 molecule can comprise, consist essentially of, or consist of 10 or more monomeric subunits (e.g., linked nucleosides), e.g., 12 or more monomeric subunits, 15 or more monomeric subunits, 18 or more monomeric subunits, 19 or more monomeric subunits, 20 or more monomeric subunits, 21 or more monomeric subunits, 22 or more monomeric subunits, 23 or more monomeric subunits, 24 or more monomeric subunits, or 25 or more monomeric subunits. Alternatively, or in addition, the miR-155 molecule can comprise, consist essentially of, or consist of 50 or less monomeric subunits, e.g., 50 or less linked monomeric subunits, 40 or less linked monomeric subunits, 30 or less monomeric subunits, 25 or less linked monomeric subunits, 24 or less monomeric subunits, 23 or less monomeric subunits, 22 or less monomeric subunits, 21 or less monomeric subunits, 20 or less linked monomeric subunits, or 19 or less linked monomeric subunits. Thus, the miR-155 molecule can comprise, consist essentially of, or consist of an amount of monomeric subunits bounded by any two of the above endpoints. For example, the miR-155 molecule can comprise, consist essentially of, or consist of 15-30 monomeric subunits, 18-25 monomeric subunits, 19-24 monomeric subunits, or 21-25 monomeric subunits. In some embodiments, the miR-155 molecule comprises, consists essentially of, or consists of 23 monomeric subunits.

In some embodiments, the miR-155 molecule is human miR-155 which comprises, consists essentially of, or consists of the sequence: UUAAUGCUAAUCGUGAUAGGGGU (SEQ ID NO: 1), or a precursor thereof. In some embodiments, the miR-155 is an analog of human miR-155 comprising a nucleic acid sequence according to SEQ ID NO: 1 except that at least one base, sugar, or internucleoside linkage has been modified, or a precursor thereof.

In other embodiments, the miR-155 molecule comprises, consists essentially of, or consists of a nucleic acid sequence which is 75% or more, e.g., 80% or more, 85% or more, 90% or more, 95% or more, 97% or more, or 99% or more, identical to SEQ ID NO: 1.

In some embodiments, the miR-155 molecule is murine miR-155 which comprises, consists essentially of, or consists of the sequence: UUAAUGCUAAUUGUGAUAGGGGU (SEQ ID NO: 2), or a precursor thereof. In some embodiments, the miR-155 molecule is an analog of murine miR-155 comprising a nucleic acid sequence according to SEQ ID NO: 2 except that at least one base, sugar, or internucleoside linkage has been modified, or a precursor thereof.

In other embodiments, the miR-155 molecule comprises, consists essentially of, or consists of a nucleic acid sequence which is 75% or more, e.g., 80% or more, 85% or more, 90% or more, 95% or more, 97% or more, or 99% or more, identical to SEQ ID NO: 2.

As used herein, the term “miRNA precursor” refers to an RNA molecule of any length which can be enzymatically processed into an miRNA, such as a primary RNA transcript, a pri-miRNA, or a pre-miRNA. The miR-155 precursor can be a pre-miR-155 which comprises, consists essentially of, or consists of 50 or more monomeric subunits (e.g., linked nucleosides), e.g., 55 or more monomeric subunits, 60 or more monomeric subunits, 65 or more monomeric subunits, 70 or more monomeric subunits, 100 or more monomeric subunits, 110 or more monomeric subunits, or 125 or more monomeric subunits. Alternatively, or in addition, the miR-155 precursor can be a pre-miR-155 which comprises, consists essentially of or consists of 150 or less monomeric subunits, e.g., 120 or less monomeric subunits, 100 or less monomeric subunits, 75 or less monomeric subunits, 70 or less monomeric subunits, 65 or less monomeric subunits, or 60 or less monomeric subunits. Thus, a pre-miR-155 can comprise, consist essentially of, or consist of an amount of monomeric subunits bounded by any two of the above endpoints. For example, a pre-miR-155 can comprise, consist essentially of, or consist of 55-100 monomeric subunits, 60-75 monomeric subunits, 60-120 monomeric subunits, or 65-70 monomeric subunits.

In some embodiments, the miR-155 molecule is a human pre-miR-155 molecule which comprises, consists essentially of, or consists of the sequence CUGUUAAUGCUAAUCGUGAUAGGGGUUUUUGCCUCCAACUGACUCCUACAUAU UAGCAUUAACAG (SEQ ID NO: 3). In other embodiments, the miR-155 molecule comprises, consists essentially of, or consists of a nucleic acid sequence which is 75% or more, e.g., 80% or more, 85% or more, 90% or more, 95% or more, 97% or more, or 99% or more, identical to SEQ ID NO: 3.

In some embodiments, the miR-155 molecule is a murine pre-miR-155 molecule which comprises, consists essentially of, or consists of the sequence CUGUUAAUGCUAAUUGUGAUAGGGGUUUUGGCCUCUGACUGACUCCUACCUG UUAGCAUUAACAG (SEQ ID NO: 4). In other embodiments, the miR-155 molecule comprises, consists essentially of, or consists of a nucleic acid sequence which is 75% or more, e.g., 80% or more, 85% or more, 90% or more, 95% or more, 97% or more, or 99% or more, identical to SEQ ID NO: 4.

In certain preferred embodiments, an exogenous nucleic acid encoding a miR-155 sequence is carried in a recombinant expression vector which contains regulatory nucleic acid sequences which provide for the miR-155 expression. The recombinant expression vector can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages.

The recombinant expression vector can be any suitable recombinant expression vector. Suitable vectors include those designed for propagation and expansion or for expression or both, such as plasmids and viruses. For example, the vector can be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors useful in the context of the invention include pBI01, pBI101.2, pBI101.3, pBI121 and pBIN19 (Clontech). Examples of animal expression vectors useful in the context of the invention include pEUK-Cl, pMAM, and pMAMneo (Clontech).

In some embodiments, the recombinant expression vector is a viral vector. Suitable viral vectors include, without limitation, retroviral vectors, alphaviral, vaccinial, adenoviral, adeno-associated viral, herpes viral, and fowl pox viral vectors, and preferably have a native or engineered capacity to transform T cells.

The recombinant expression vectors can be prepared using standard recombinant DNA techniques described in, for example, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector can comprise regulatory sequences, such as transcription and translation initiation and termination codons, which are specific to the type of host (e.g., bacterium, fungus, plant, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the recombinant expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleic acid encoding the miR-155 molecule. Preferably, the promoter is functional in T cells. The selection of a promoter, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vector can be designed for either transient expression, for stable expression, or for both. Also, the recombinant expression vectors can be made for constitutive expression or for inducible expression.

The vectors useful in the context of the invention can be “naked” nucleic acid vectors (i.e., vectors having little or no proteins, sugars, and/or lipids encapsulating them), or vectors complexed with other molecules. Other molecules that can be suitably combined with the vectors include without limitation viral coats, cationic lipids, liposomes, polyamines, gold particles, and targeting moieties such as ligands, receptors, or antibodies that target cellular molecules.

Preferably, a CD8⁺ T cell comprising an antigen-specific TCR is isolated or purified as described herein, and then contacted with an exogenous nucleic acid encoding a miR-155 molecule ex vivo or in vitro using methods described herein or any other method known in the art. Examples of such means include, but are not limited to, the use of a lipid, protein, particle, or other molecule capable of facilitating cell transformation with the nucleic acid. However, a CD8⁺ T cell comprising an antigen-specific TCR also can be contacted with an exogenous nucleic acid encoding a miR-155 molecule in vivo, such as by way of a gene gun, for example. Suitable methods of administering a vector of the invention to a mammal for purposes of gene therapy are known (see, e.g., Rosenfeld et al., Science, 252: 431-434 (1991); Jaffe et al., Clin. Res., 39: 302A (1991); Rosenfeld et al., Clin. Res., 39: 311A (1991); Berkner, BioTechniques, 6: 616-629 (1988); Crystal et al., Human Gene Ther., 6: 643-666 (1995); Crystal et al., Human Gene Ther., 6: 667-703 (1995)).

The isolated or purified CD8⁺ T cell of the invention which has been contacted with an exogenous nucleic acid encoding a miR-155 molecule preferably expresses the exogenous miR-155 molecule efficiently. For example, without limiting the invention, a CD8⁺ T cell or a population thereof expressing an exogenous miR-155 molecule can contain an amount of miR-155 that is 1.5-fold higher or more, e.g., 2-fold higher or more, 3-fold higher or more, 5-fold higher or more, 10-fold higher or more, 20-fold higher or more, or 50-fold higher or more, than the amount of miR-155 present in a control CD8⁺ T cell or a population thereof not expressing an exogenous miR-155 molecule. Alternatively, or in addition, the CD8⁺ T cell or a population thereof expressing an exogenous miR-155 molecule can contain an amount of miR-155 that is 100-fold higher or less, e.g., 80-fold higher or less, 60-fold higher or less, 30-fold higher or less, 15-fold higher or less, 8-fold higher or less, or 4-fold higher or less, than the amount of miR-155 present in a control CD8⁺ T cell or a population thereof not expressing an exogenous miR-155 molecule. Thus, the miR-155 can be present in a CD8⁺ T cell or population thereof in an amount bounded by any two of the above endpoints. For example, the CD8⁺ T cell or a population thereof expressing an exogenous miR-155 molecule can contain an amount of miR-155 that is 1.5-15-fold higher, 2-4-fold higher, 3-30-fold higher, 5-8-fold higher, or 10-100-fold higher, than the amount of miR-155 present in a control CD8⁺ T cell or a population thereof not expressing an exogenous miR-155 molecule. Any suitable method known in the art can be utilized to determine the amount of miR-155 present in a CD8⁺ T cell or a population thereof, such as quantitative RT-PCR or stem-loop quantitative RT-PCR (see, e.g., Chen et al., Nucl. Acids Res., 33 (20): e179 (2005)).

The invention also provides a method of treating a medical condition, e.g., a disease in a mammal. The method comprises administering to the mammal any of the CD8⁺ T cells described herein, or a population thereof, or a composition comprising any of the CD8⁺ T cells described herein, in an amount effective to treat the medical condition in the mammal.

The invention further provides a method of preventing a medical condition, e.g., a disease in a mammal. The method comprises administering to the mammal any of the CD8⁺ T cells described herein, or a population thereof, or a composition comprising any of the CD8⁺ T cells described herein, in an amount effective to prevent the medical condition in the mammal.

The medical condition to be treated or prevented by the inventive methods include any of the medical conditions or diseases for which the TCR of the CD8⁺ T cell of the invention is antigen-specific. For example, the disease or medical condition can be a cancer, an infectious disease, an autoimmune disease, or an immunodeficiency, as discussed hereinabove.

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect.

The term “mammal” as used herein refers to any mammal, including, but not limited to, mice, hamsters, rats, rabbits, cats, dogs, cows, pigs, horses, monkeys, apes, and humans. Preferably, the mammal is a human.

Preferably, the medical condition to be treated or prevented is cancer. In certain embodiments, the cancer is melanoma. Thus, the invention provides a method of reducing the size of a tumor in a mammal which comprises administering to the mammal any of the CD8⁺ T cells described herein, or a population thereof, or a composition comprising any of the CD8⁺ T cells described herein, in an amount effective to reduce the size of the tumor in the mammal. In some embodiments, the method effectively treats cancer in the mammal.

In the treatment or prevention of a medical condition, e.g., a disease, in a mammal, the CD8⁺ T cells that have been transformed, e.g., transduced, with an exogenous nucleic acid encoding a miR-155 molecule can be transferred into the same mammal from which CD8⁺ T cells were obtained. In other words, the CD8⁺ T cell used in the inventive method of treating or preventing can be an autologous CD8⁺ T cell, i.e., can be obtained from the mammal in which the medical condition is treated or prevented. Alternatively, the CD8⁺ T cell can be allogenically transferred into another mammal. Preferably, the T CD8⁺ T cell is autologous to the mammal in the inventive method of treating or preventing a medical condition in the mammal.

In the instance that the CD8⁺ T cells are autologous to the mammal, the mammal can be immunologically naïve, immunized, diseased, or in another condition prior to isolation of the CD8⁺ T cells from the mammal. In some instances, it is preferable for the method to comprise immunizing the mammal with an antigen of the medical condition prior to isolating the CD8⁺ T cell from the mammal, contacting (e.g., transducing or transfecting) the obtained CD8⁺ T cell with the exogenous nucleic acid encoding a miR-155 molecule, and the administering of the CD8⁺ T cell, or a population or composition thereof. As discussed herein, immunization of the mammal with the antigen of medical condition will allow the population of CD8⁺ T cells having an endogenous TCR reactive with the medical condition-specific antigen to increase in numbers, which will increase the likelihood that the CD8⁺ T cell obtained for contacting with the nucleic acid encoding miR-155 will have a desired antigen-specific TCR.

In accordance with the invention, a mammal with a medical condition can be therapeutically immunized with an antigen from, or associated with, that medical condition, including immunization via a vaccine. While not desiring to be bound by any particular theory, the vaccine or immunogen is provided to enhance the mammal's immune response to the medical condition antigen present in or on the infectious agent or diseased tissue. Such a therapeutic immunization includes, but is not limited to, the use of recombinant or natural disease proteins, peptides, or analogs thereof, or modified disease peptides, or analogs thereof that can be used as a vaccine therapeutically as part of adoptive immunotherapy. The vaccine or immunogen, can be a cell, cell lysate (e.g., from cells transfected with a recombinant expression vector), a recombinant expression vector, or antigenic protein. Alternatively, the vaccine, or immunogen, can be a partially or substantially purified recombinant disease protein, peptide or analog thereof, or modified peptides or analogs thereof. The proteins or peptides may be conjugated with lipoprotein or administered in liposomal form or with adjuvant. Preferably, the vaccine comprises one or more of (i) the medical condition-antigen for which the TCR of the CD8⁺ T cell of the invention is specific, (ii) an epitope of the antigen, and (iii) a vector encoding the antigen or the epitope.

The inventive method of treating or preventing a medical condition in a mammal can comprise additional steps. For instance, a variety of procedures, as discussed below, can be performed on the CD8⁺ T cells prior to, substantially simultaneously with, or after their isolation from a mammal. Similarly, a variety of procedures can be performed on the CD8⁺ T cells prior to, substantially simultaneously with, or after their contacting with an exogenous nucleic acid encoding a miR-155 molecule.

Preferably, the CD8⁺ T cells are expanded in vitro after contacting (e.g., transducing or transfecting) the cells with an exogenous nucleic acid encoding a miR-155 molecule, but prior to the administration to a mammal. In vitro expansion can proceed for 1 day or more, e.g., 2 days or more, 3 days or more, 4 days or more, 6 days or more, or 8 days or more, prior to the administration to a mammal. Alternatively, or in addition, in vitro expansion can proceed for 21 days or less, e.g., 18 days or less, 16 days or less, 14 days or less, 10 days or less, 7 days or less, or 5 days or less, prior to the administration to a mammal. Thus, in vitro expansion can proceed for a duration bounded by any two of the above endpoints. For example, in vitro expansion can proceed for 1-7 days, 2-10 days, 3-5 days, or 8-14 days prior to the administration to a mammal.

During in vitro expansion, the CD8⁺ T cells can be stimulated with the medical condition-antigen for which the TCR is specific. Antigen specific expansion optionally can be supplemented with expansion under conditions that non-specifically stimulate lymphocyte proliferation such as, for example, anti-CD3 antibody, anti-Tac antibody, anti-CD28 antibody, or phytohemagglutinin (PHA). The expanded CD8⁺ T cells can be directly administered into the mammal or can be frozen for future use, i.e., for subsequent administrations to a mammal.

Currently, in many ACT-based cancer treatment regimens, an isolated T cell population is treated ex vivo with the T cell growth factor interleukin-2 (IL-2) prior to infusion into a cancer patient, and the cancer patient is treated with IL-2 after infusion. Furthermore, the cancer patient typically undergoes preparative lymphodepletion—the temporary ablation of the immune system—prior to ACT. The combination of IL-2 treatment and preparative lymphodepletion is associated with enhanced persistence of the transferred T cells and can lead to prolonged tumor eradication (Restifo et al., supra).

In accordance with the invention, it is not required that a mammal receiving a CD8⁺ T cell comprising an exogenous nucleic acid encoding a miR-155 molecule is administered IL-2, or any other cytokine which signals through the IL-2 receptor gamma (also known as common gamma chain (γc), IL-2RG, and CD132). Cytokines which signal through the IL-2 receptor gamma include, for example, IL-2, IL-4, IL-7, IL-9, IL-15, and IL-21. Without wishing to be bound to any particular theory, it is believed that the exogenous miR-155 provides a proliferative and/or survival benefit to the CD8⁺ T cell of the invention which bypasses the requirement for IL-2 administration to enhance the T cell expansion and/or survival following ACT. Thus, in certain embodiments of the inventive method of treating or preventing a medical condition in a mammal, the method does not comprise administering to the mammal IL-2 or another cytokine which signals through the IL-2 receptor gamma prior to, substantially simultaneously with, or after administration of a CD8⁺ T cell of the invention.

However, in certain embodiments cytokines desirably are contacted to the CD8⁺ T cell of the invention ex vivo, or administered to a mammal receiving the CD8⁺ T cell of the invention. In some embodiments, a CD8⁺ T cell of the invention can be transduced or transfected with a nucleic acid encoding a cytokine, which nucleic acid can be engineered to provide for constitutive, regulatable, or temporally-controlled expression of the cytokine. Suitable cytokines include, for example, cytokines which act to enhance the survival of T lymphocytes during the contraction phase, which can facilitate the formation and survival of memory T lymphocytes.

In certain embodiments of the inventive treatment and prevention methods, the CD8⁺ T cell is administered prior to, substantially simultaneously with, or after the administration of another therapeutic agent, such as a cancer therapeutic agent. The cancer therapeutic agent can be a chemotherapeutic agent, a biological agent, or radiation treatment. However, in certain embodiments, of the inventive treatment and prevention methods, the mammal receiving the CD8⁺ T cell of the invention is not administered a treatment which is sufficient to cause a depletion of immune cells, such as lymphodepleting chemotherapy or radiation therapy.

The CD8⁺ T cell, or populations thereof, of the invention, can be formed as a composition. Thus, the invention provides a composition comprising at least one CD8⁺ T cell of the invention, and a carrier therefor. In certain embodiments, the composition is a pharmaceutical composition comprising at least one CD8⁺ T cell of the invention and a pharmaceutically acceptable carrier, diluent, and/or excipient.

The pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., the CD8⁺ T cell, and does not elicit any detrimental side effects or toxicity under the conditions of use.

The composition can be formulated for administration by any suitable route, such as, for example, an administration route selected from the group consisting of intravenous, intratumoral, intraarterial, intramuscular, intraperitoneal, intrathecal, epidural, and subcutaneous administration routes. Preferably, the composition is formulated for a parenteral route of administration.

A composition suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

The dose administered to a mammal, particularly a human, in the context of the invention will vary with the inventive embodiment, the composition employed, the method of administration, and the particular site and mammal being treated. However, the dose should be sufficient to provide a therapeutic response. As used herein, an “effective amount” or “therapeutically effective amount” refers to an amount that relieves (to at least some extent) one or more symptoms of a medical condition in a human or other mammalian subject. Additionally, an “effective amount” or “therapeutically effective amount” refers to an amount that returns to normal, either partially or completely, physiological or biochemical parameters associated with or causative of the medical condition. A clinician skilled in the art can determine the therapeutically effective amount of a composition to be administered to a human or other mammalian subject in order to treat or prevent a particular medical condition. The precise amount of the composition required to be therapeutically effective will depend upon numerous factors, e.g., such as the specific activity of the CD8⁺ T cell of the invention, and the route of administration, in addition to many patient-specific considerations.

Any suitable number of CD8⁺ T cells of the invention can be administered to a mammal. While a single CD8⁺ T cell of the invention theoretically is capable of expanding and providing a therapeutic benefit, it is preferable to administer 10² or more, e.g., 10³ or more, 10⁴ or more, 10⁵ or more, 10⁸ or more, CD8⁺ T cells of the invention. Alternatively, or additionally 10¹² or less, e.g., 10¹¹ or less, 10⁹ or less, 10⁷ or less, or 10⁵ or less, CD8⁺ T cells of the invention can be administered to a mammal. The number of CD8⁺ T cells of the invention can be administered to a mammal in an amount bounded by any two of the above endpoints, e.g., 10²-10⁵, 10⁴-10⁷, 10³-10⁹, or 10⁵-10¹⁰.

A dose of the CD8⁺ T cell of the invention can be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a CD8⁺ T cell of the invention may be administered in a single daily dose, or the total daily dosage may be administered in two, three, four, or more divided doses administered daily, as needed.

Although there is no theoretical upper limit on the number of CD8⁺ T cells of the invention that can be administered to a mammal or the number of times that the CD8⁺ T cells of the invention can be administered to a mammal, one of ordinary skill in the art will understand that excessive quantities of administered T lymphocytes can lead to undesirable side effects and unnecessarily increase costs.

In some embodiments, a pharmaceutical composition comprising at least one CD8⁺ T cell of the invention does not substantially contain any other living cells. In other embodiments, a pharmaceutical composition comprises at least one CD8⁺ T cell of the invention as well as other CD8⁺ T cells which do not comprise an antigen-specific TCR and/or which do not comprise an exogenous nucleic acid encoding a miR-155 molecule. In yet other embodiments, a pharmaceutical composition comprises at least one CD8⁺ T cell of the invention as well as other blood cells (e.g., lymphocytes) which may or may not comprise an exogenous nucleic acid encoding a miR-155 molecule. Such pharmaceutical compositions can be readily prepared by positive and/or negative selection of the desired cells from a population of cells contacted with an exogenous nucleic acid encoding a miR-155 molecule. Suitable positive and negative selection techniques are well known in the art and include, for example, flow cytometry and immunomagnetic separation. Negative selection also can comprise the use of antibiotics to destroy microbes. Moreover, leukophoresis, other filtration techniques, sterile technique, differential centrifugation, and other conventional methods can be used to produce a composition suitable for administration to a human.

A pharmaceutical composition comprising the CD8⁺ T cell of the invention can optionally contain one or more additional therapeutic agents, such as a cancer therapeutic agent, e.g., a chemotherapeutic agent or a biological agent.

Examples of chemotherapeutic agents which can be used in the compositions and methods of the invention include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents which can be used in the compositions and methods of the invention include monoclonal antibodies (e.g., rituximab, cetuximab, panetumumab, tositumomab, trastuzumab, alemtuzumab, gemtuzumab ozogamicin, and bevacizumab), enzymes (e.g., L-asparaginase), cytokines (e.g., interferons and interleukins), growth factors (e.g., colony stimulating factors and erythropoietin), cancer vaccines, gene therapy vectors, or any combination thereof.

The treatment methods of the invention can be performed on mammals for which other treatments of the medical condition have failed or have had less success in treatment through other means. Also, the treatment methods of the invention can be performed in conjunction with other treatments of the medical condition. For instance, the method can comprise administering a cancer regimen, e.g., nonmyeloablative chemotherapy, surgery, hormone therapy, and/or radiation, prior to, substantially simultaneously with, or after the administration of the CD8⁺ T cell of the invention, or population or composition thereof. In certain embodiments, a mammal to which the CD8⁺ T cells of the invention are administered can also be treated with antibiotics or other pharmaceutical agents.

The following examples further illustrate the invention but, of course, should not be construed as in any way limiting its scope.

### EXAMPLE 1

This example demonstrates the preparation of an isolated CD8⁺ T cell which contains an antigen-specific TCR and an exogenous nucleic acid encoding a miR-155 molecule.

Pmel-1 transgenic mice produce T cells expressing a TCR with specificity for gp100, which is an enzyme expressed by malignant melanoma cells as well as normal melanocytes (Overwijk et al., J. Exp. Med., 198 (4): 569-580 (2003)). In pmel-1 transgenic mice, greater than 95% of CD8⁺ T cells express a TCR which recognizes an H-2D^{b}-restricted epitope corresponding to amino acids 25-33 of gp100 of human and mouse origin (Overwijk et al., supra).

CD8⁺ T cells were isolated from the spleens of pmel-1 transgenic mice using the CD8a⁺ T cell Isolation Kit II (Miltenyi Biotec, Auburn, Calif.), according to the manufacturer's instructions. The CD8⁺ T cells were stimulated with anti-CD3 (2 μg/mL) and anti-CD28 (1 μg/mL) antibodies for 24 hours, and then transduced with a retrovirus encoding GFP linked to a nucleic acid encoding murine miR-155 or a control, scrambled version of murine miR-155 (referred to herein as “scramble” or “scramble miR”). The core retrovirus used for expression of murine miR-155 and scrambled miR-155 has been described previously (O'Connell et al., Proc. Natl. Acad. Sci. U.S.A., 106 (17): 7113-7118 (2009)). The murine miR-155 was encoded by a murine pre-miR-155 according to SEQ ID NO: 4.

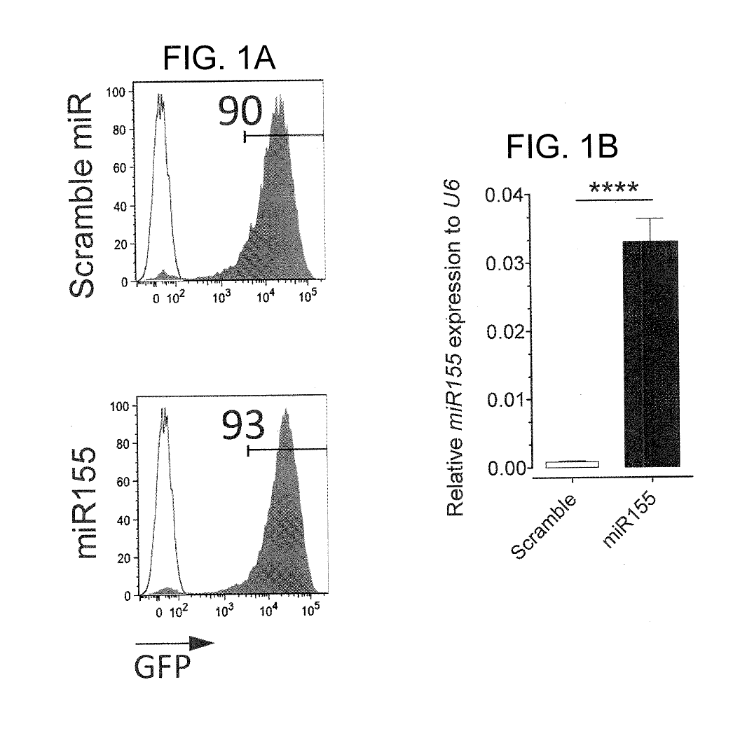

CD8⁺ T cells transduced with retrovirus encoding miR-155 or scrambled miR-155 displayed similar levels of GFP expression, as assessed by FACS analysis (FIG. 1A). CD8⁺ T cells transduced with retrovirus encoding miR-155 expressed significantly higher amounts of miR-155 than CD8⁺ T cells transduced with retrovirus encoding scrambled miR-155, as assessed by qPCR analysis (FIG. 1B).

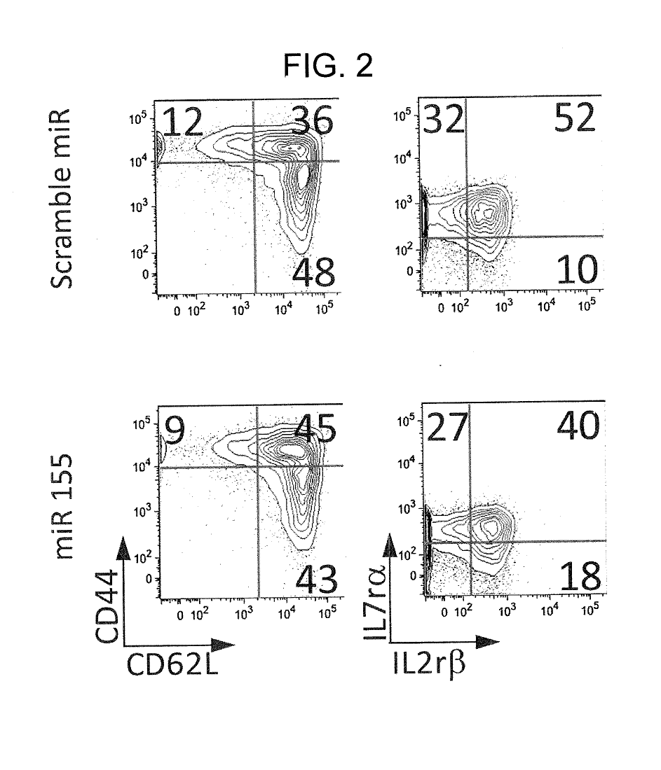

Overexpression of miR-155 in CD8⁺ T cells did not affect differentiation status, as demonstrated by the similar levels of expression of CD44, CD62L, IL7rα, and IL7rβ observed in CD8⁺ T cells transduced with retrovirus encoding miR-155 or scrambled miR-155 after 5 days of culture in vitro (FIG. 2). CD8⁺ T cells expressing miR-155 or scrambled miR-155 both displayed a CD62L⁺CD44⁺ central memory phenotype (FIG. 2).

The results of this example provide an isolated CD8⁺ T which contains (a) a TCR specific for gp100 and (b) an exogenous nucleic acid encoding murine miR-155.

### EXAMPLE 2

This example demonstrates a method of reducing the size of a tumor in a mammal by administering a population of CD8⁺ T cells expressing exogenous miR-155.

B16 is a spontaneous murine melanoma which expresses gp100, and adoptive transfer of CD8⁺ T cells expressing a gp100-specific TCR into mice bearing an B16 tumor can result in tumor destruction in vivo (Overwijk et al., J. Exp. Med., 188: 277-286 (1998)).

The B16 melanoma model was used to assess the role of miR-155 in CD8⁺ T cells during tumor destruction according to standard protocols. Briefly, CD8⁺ T cells isolated from pmel-1 transgenic mice as described in Example 1 were transduced with retrovirus encoding miR-155 or scrambled miR-155, and expanded in vitro for 4 days. 8×10⁶ CD8⁺ T cells were intravenously injected into C57BL/6 mice bearing a B16 tumor approximately 50 mm² in size. Tumors were measured using calipers in a blinded fashion at various time points following ACT, and the products of perpendicular tumor diameters were recorded. Mice were sacrificed once tumors reached 300-400 mm² in size. In certain experiments, the CD8⁺ T cells were injected in conjunction with intravenous administration of a gp100 vaccination (2×10⁷ pfu of a recombinant vaccina virus encoding human gp100 (rvvhgp100) at the time of CD8⁺ T cell injection).

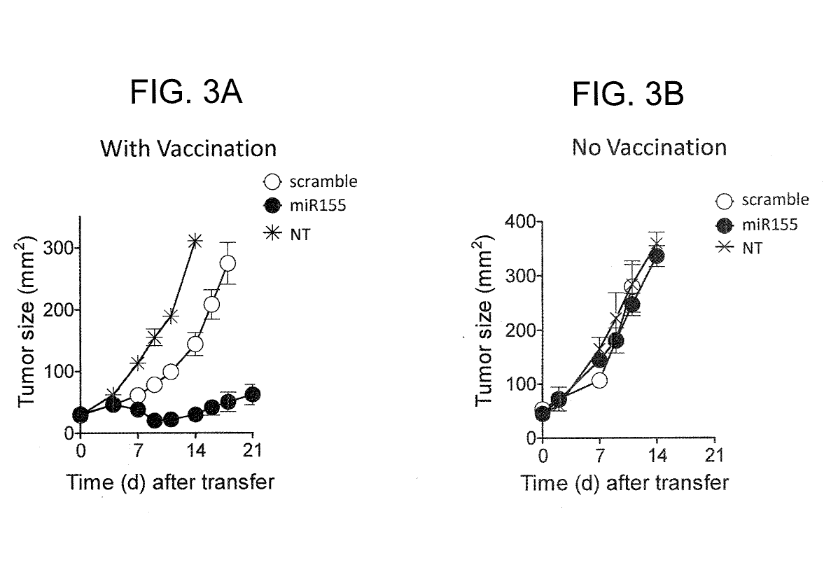

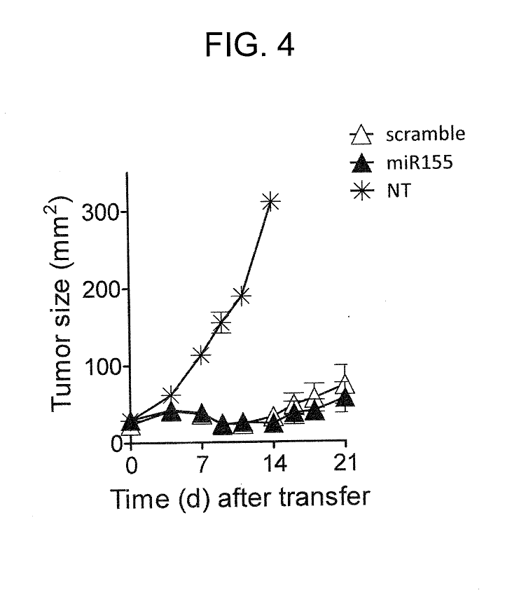

B16 tumors grew rapidly in gp100-vaccinated mice which did not receive CD8⁺ T cells. The growth of B16 tumors in gp100-vaccinated mice which received CD8⁺ T cells overexpressing scrambled miR-155 was slightly delayed. In contrast, the growth of B16 tumors in gp100-vaccinated mice which received CD8⁺ T cells overexpressing miR-155 was markedly inhibited (FIG. 3A). None of the mice used in the experiments depicted in FIG. 3A were irradiated prior to ACT, and none received exogenous IL-2 after ACT, thereby demonstrating that miR-155 expression in CD8⁺ T cells inhibits tumor growth in the absence of lymphodepletion or exogenous IL-2 administration. However, vaccination of mice bearing B16 tumors was necessary for CD8⁺ T cells overexpressing miR-155 to mediate efficacious tumor growth inhibition, as demonstrated by the absence of tumor growth inhibition in mice not administered with rvvhgp100 (FIG. 3B).

To determine the effect of IL-2 supplementation, tumor growth was measured in gp100-vaccinated mice which received CD8⁺ T cells overexpressing miR-155 or scrambled miR-155 and exogenous IL-2 (6×10⁴ Cetus units (CU) immediately following CD8⁺ T cell injection, and at 12 hour intervals thereafter for a total of 6 doses). Tumor growth was markedly inhibited in mice which received CD8⁺ T cells overexpressing miR-155 or scrambled miR-155 when the mice also received exogenous IL-2, suggesting that exogenous IL-2 abrogates the advantage of miR-155 overexpression in CD8⁺ T cells with respect to B16 growth inhibition in this model (FIG. 4).

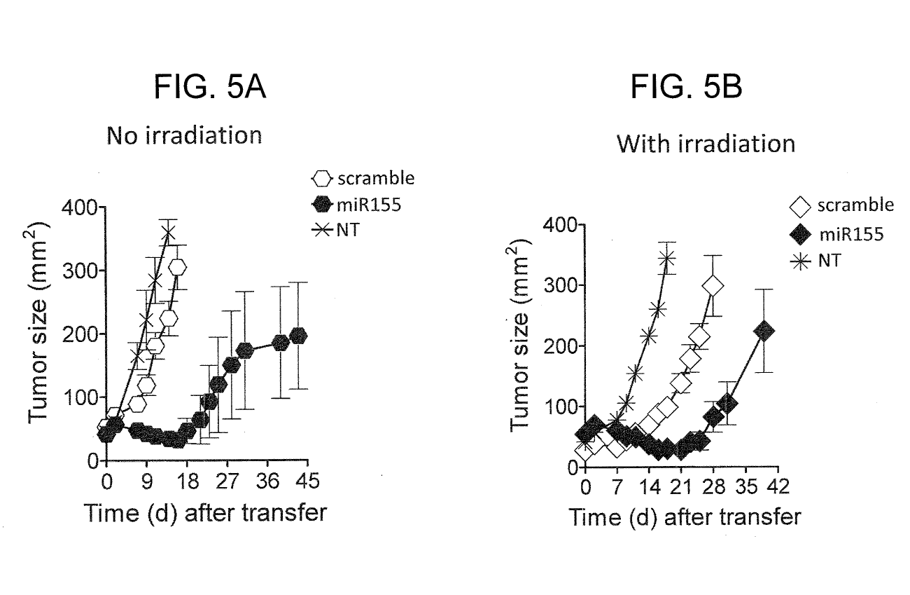

To determine the effect of preparative lymphodepletion, tumor growth was measured in gp100-vaccinated mice which were irradiated prior to ACT (6 Gy, approximately 30 minutes prior to CD8⁺ T cell injection). Tumor growth was slightly delayed in non-irradiated mice which received CD8⁺ T cells overexpressing scrambled miR-155 (FIG. 5A), and the degree of inhibition was greater in irradiated mice (FIG. 5B). Tumor growth was markedly inhibited in non-irradiated mice which received CD8⁺ T cells overexpressing miR-155 (FIG. 5A), and irradiation treatment did not substantially further the degree of growth inhibition (FIG. 5B). At all time points beyond approximately 9 days following ACT, tumor growth inhibition was greater in mice which received CD8⁺ T cells overexpressing miR-155 as compared to mice which received CD8⁺ T cells overexpressing scrambled miR-155, irrespective of whether the mice had been irradiated prior to ACT.

The results of this example demonstrate that adoptive transfer of CD8⁺ T cells overexpressing miR-155 in a tumor bearing mammal reduces tumor growth in an exogenous IL-2- and preparative lymphodepletion-independent manner.

### EXAMPLE 3

This example demonstrates that adoptive transfer of CD8⁺ T cells overexpressing miR-155 results in tumor growth inhibition in various immunodeficient mouse host strains.

Mice deficient in CD4 have defective helper T cell activity and other T cell responses, whereas mice deficient in CD8 have defective cytotoxic T cell responses. Mice deficient in recombination activating gene 1 (RAG-1) have defective B cell and T cell development, and produce no mature B cells or mature T cells.

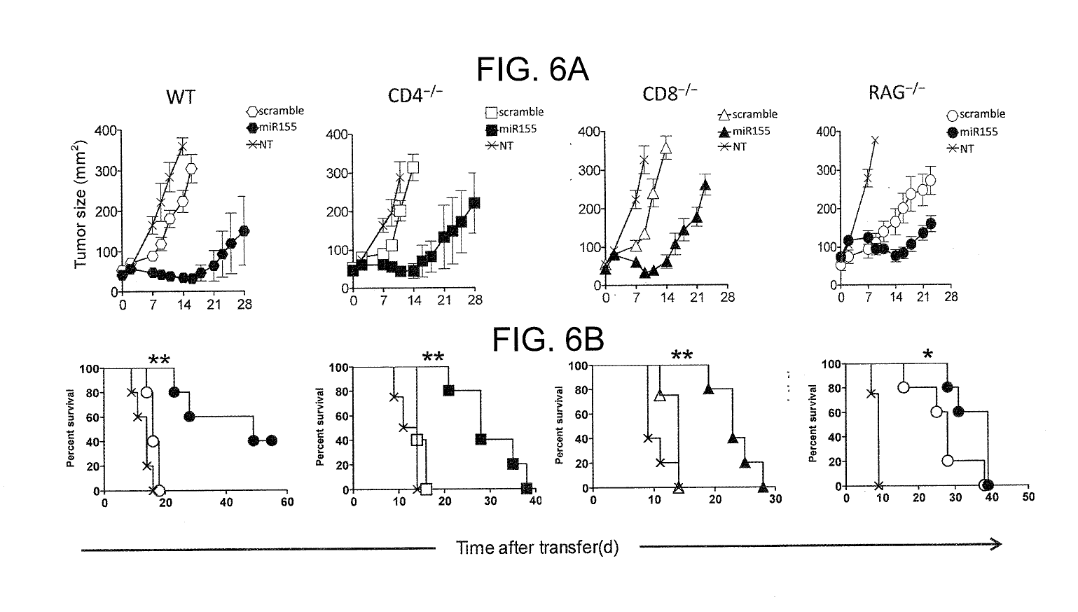

To determine the effect of host immune system on tumor growth inhibition, CD8⁺ T cells overexpressing miR-155 or scrambled miR-155 were injected into wild-type, CD4^{-/-}, CD8^{-/-}, or RAG-1^{'1/-} mice vaccinated with rvvhgp100, and tumor growth was monitored as described in Example 2. Adoptive transfer of CD8⁺ T cells overexpressing miR-155 resulted in substantially greater tumor growth inhibition as compared to scrambled miR-155 in each of the tested strains of mice (FIG. 6A), which was correlated with a statistically significant increase in duration of survival (FIG. 6B).

The results of this example demonstrate that adoptive transfer of CD8⁺ T cells overexpressing miR-155 reduces tumor growth in genetically immunodeficient mammals.

### EXAMPLE 4

This example demonstrates that miR-155 overexpressing CD8⁺ T cells expand more and contract less as compared to control CD8⁺ T cells following ACT.

The number of CD8⁺ T cells expressing a TCR with specificity for gp100 peaks approximately 4-5 days following ACT and rvvhgp100 vaccination (Overwijk et al. (2003), supra). To determine whether miR-155 overexpression affects T cell expansion or contraction, splenocytes were harvested from wild-type C57BL/6 mice at 4, 5, 6, or 7 days following ACT with CD8⁺ T cells overexpressing miR-155 or scrambled miR-155 in conjunction with rvvhgp100 vaccination, and assayed for GFP and CD8 production by FACS.

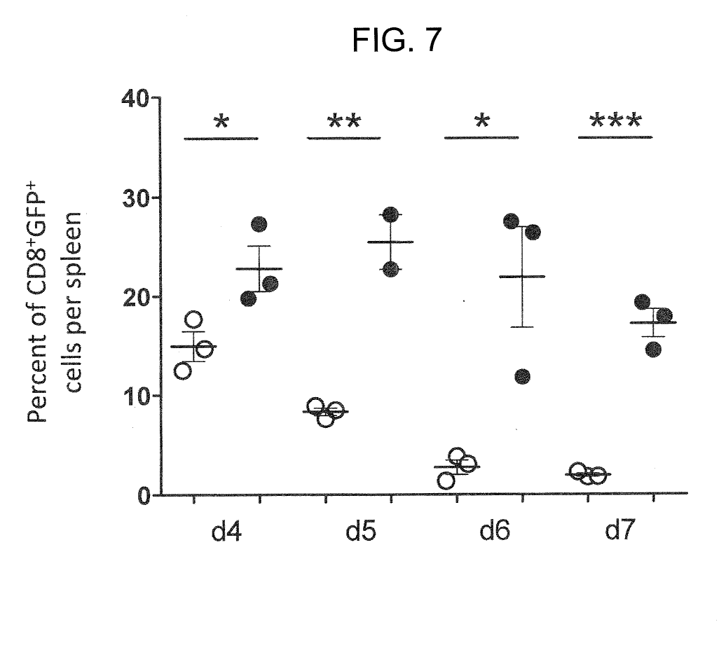

There was a significantly greater percentage of CD8⁺GFP⁺ cells in the spleens of mice which had received CD8⁺ T cells overexpressing miR-155 as compared to mice which had received CD8⁺ T cells overexpressing scrambled miR-155 at each of 4, 5, 6, and 7 days following ACT (FIG. 7).

These results demonstrate that overexpression of miR-155 in CD8⁺ T cells promotes cell expansion and delays cell contraction following ACT.

### EXAMPLE 5

This example demonstrates that miR-155 overexpressing CD8⁺ T cells display a greater persistence of cytokine production as compared to control CD8⁺ T cells following ACT.

To determine whether miR-155 overexpression affects T cell cytokine production, splenocytes were harvested from wild-type C57BL/6 mice at 4 or 6 days following ACT with CD8⁺ T cells overexpressing miR-155 or scrambled miR-155 in conjunction with rvvhgp100 vaccination, and assayed for IFN-γ, IL-2, and TNF-α production by FACS.

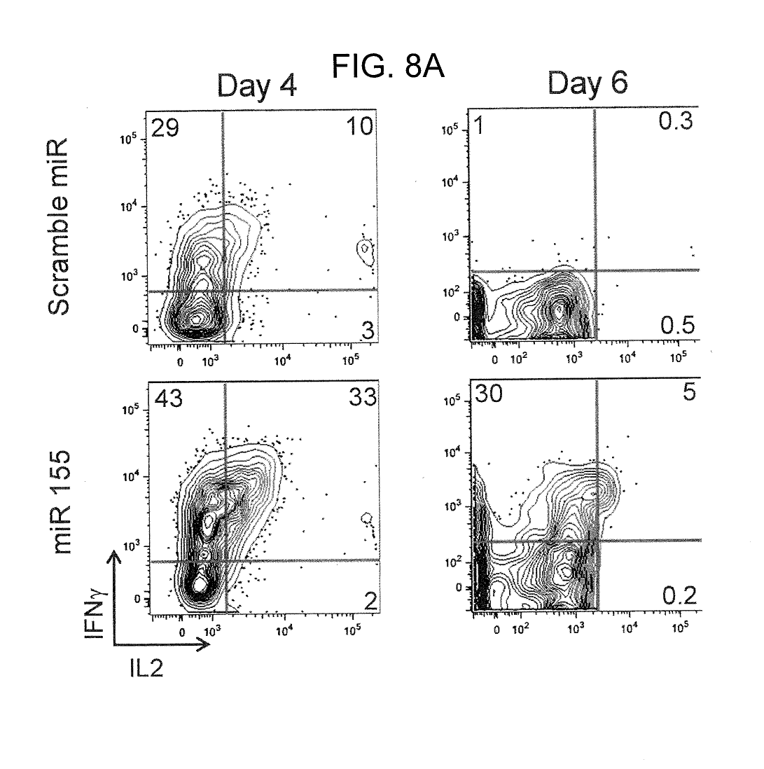

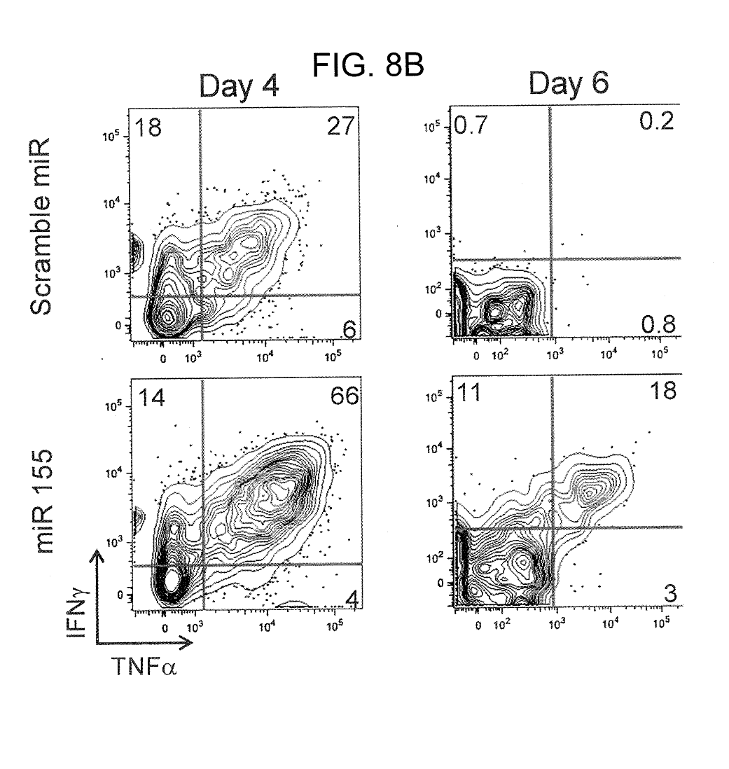

There were greater numbers of IFN-γ⁺IL-2⁺ cells in the spleens of mice which had received CD8⁺ T cells overexpressing miR-155 as compared to mice which had received CD8⁺ T cells overexpressing scrambled miR-155 at 4 and 6 days following ACT (FIG. 8A). Similarly, the numbers of IFN-γ⁺TNF-α⁺ cells in the spleens of mice which had received CD8⁺ T cells overexpressing miR-155 were greater when compared with mice which had received CD8⁺ T cells overexpressing scrambled miR-155 at 4 and 6 days following ACT (FIG. 8B).

These results demonstrate that overexpression of miR-155 in CD8⁺ T cells leads to a slower rate of “shut-down” of IFN-γ, IL-2, and TNF-α production following ACT.

### EXAMPLE 6

This example demonstrates the role of homeostatic cytokines in the functionality of miR-155 overexpressing CD8⁺ T cells adoptively transferred to mice.

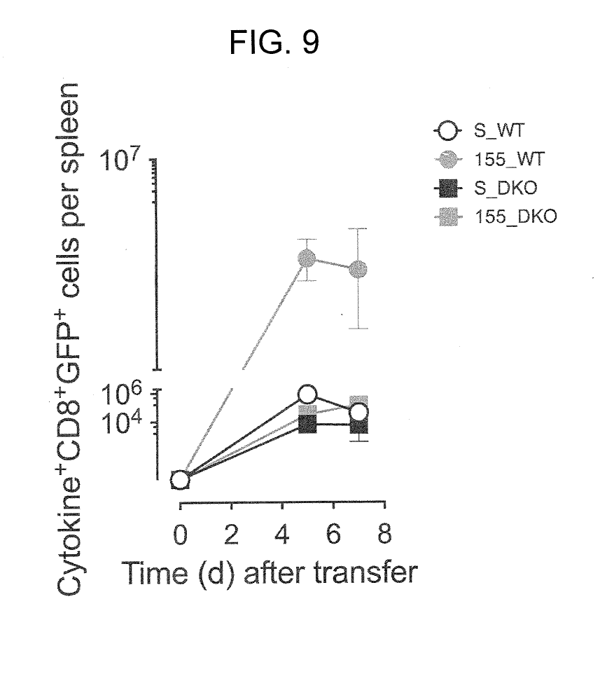

5×10⁵ pmel-1 CD8⁺ T cells overexpressing miR-155 or scrambled miR as described in Example 1 were intravenously injected into wild-type C57BL/6 mice or mice deficient of IL-7 and IL-15 (Il-7^{-/'1}Il-15^{-/-}) infected with rvvhgp100. Mice were assessed 0-7 days after infection with rvvhgp100 and assayed for GFP and CD8 percentage by FACS. The functionality of miR-155 overexpressing CD8⁺ T cells was impaired in Il-7^{-/-}Il-15^{-/-} mice, as shown in FIG. 9.

The results of this example demonstrates that the functionality of miR-155-overexpressing CD8⁺ T cells is impaired in mice deficient in certain homeostatic cytokines, suggesting that miR-155 acts through these homeostatic cytokines.

### EXAMPLE 7

This example demonstrates that miR-155 is dynamically regulated in CD8⁺ T cells depending on the magnitude of TCR stimulation or their differentiation state.

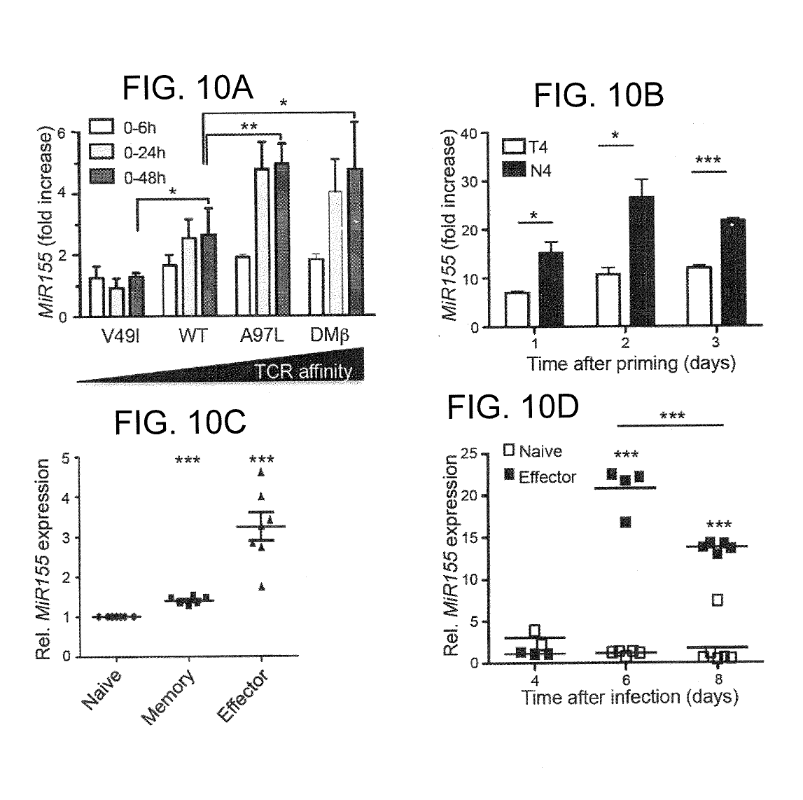

The strength of TCR signaling has a major impact on the magnitude of CD8⁺ T cell expansion but not on their differentiation (see, e.g., Zehn et al., Nature, 458: 211-214 (2009)). As such, the effects of TCR stimulation strength on the expression of miR-155 in CD8⁺ T cells was investigated. Human CD8⁺ T cells were transduced with variants of a NY-ESO-1-specific TCR of increasing affinity for its ligand (see, e.g., Derre et al., Proc. Natl. Acad. Sci. USA, 105: 15010-15015 (2008); and Schmid et al., J. Immunol., 184: 4936-4946 (2010)). Comparatively, a mutated low affinity TCR failed to upregulate miR-155 within 48 hours, whereas TCR variants of higher affinities induced higher levels of miR-155 than the wild-type TCR, as shown in FIG. 10A. Thus, miR-155 expression increased in a TCR affinity-dependent manner in human CD8⁺ T cells. A similar upregulation was observed for the pri-miR-155 non-coding RNA transcript, BIC.

To determine whether miR-155 was also regulated in an affinity-dependent manner in mouse CD8⁺ T cells, naïve OT-1 T cells were activated with splenic dendritic cells (DC) loaded with the wild-type peptide SIINFEKL (SEQ ID NO: 5) or the weaker altered peptide ligand SIITFEKL (SEQ ID NO: 6) (Daniels et al., Nature, 444: 724-729 (2006)). To exclude miR-155 contamination from the DCs, miR-155-deficient DC (Mir155^{-/-}) which retain normal antigen presenting capabilities were used (O'Connell et al., Immunity, 33: 607-619 (2010)). Exposure of OT-1 cells to the wild-type natural peptide resulted in a strong upregulation of miR-155, while a weaker TCR stimulation by the altered peptide ligand was less effective, as shown in FIG. 10B. To assess miR-155 regulation in vivo, naïve (CD62L⁺CD44⁻), effector (CD62L⁻CD44⁺), and central memory (CD62L⁺CD44⁺) CD8⁺ T cells were analyzed following infection with lymphocytic choriomeningitis virus (LCMV) (200 pfu of the WE strain). Compared to their naïve counterparts, miR-155 was strongly upregulated in effector cells and to a lower extent in central memory CD8⁺ T cells 8 days post-infection, as shown in FIG. 10C. A more detailed kinetics of miR-155 regulation during LCMV infection revealed that numbers of effector cells peaked on day six, but stayed low in naïve cells, as shown in FIG. 10D.

The results of this example demonstrate that miR-155 is induced in effector CD8⁺ T cells depending on the strength of stimulation and differentiation.

### EXAMPLE 8

This example demonstrates that miR-155 promotes the accumulation of anti-viral effector and central memory CD8⁺ T cells.

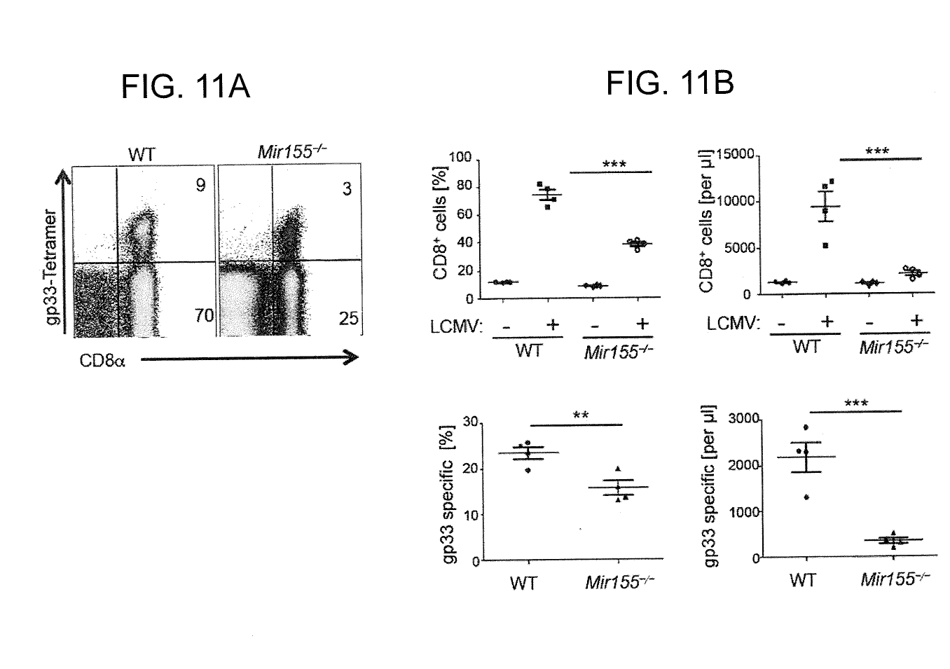

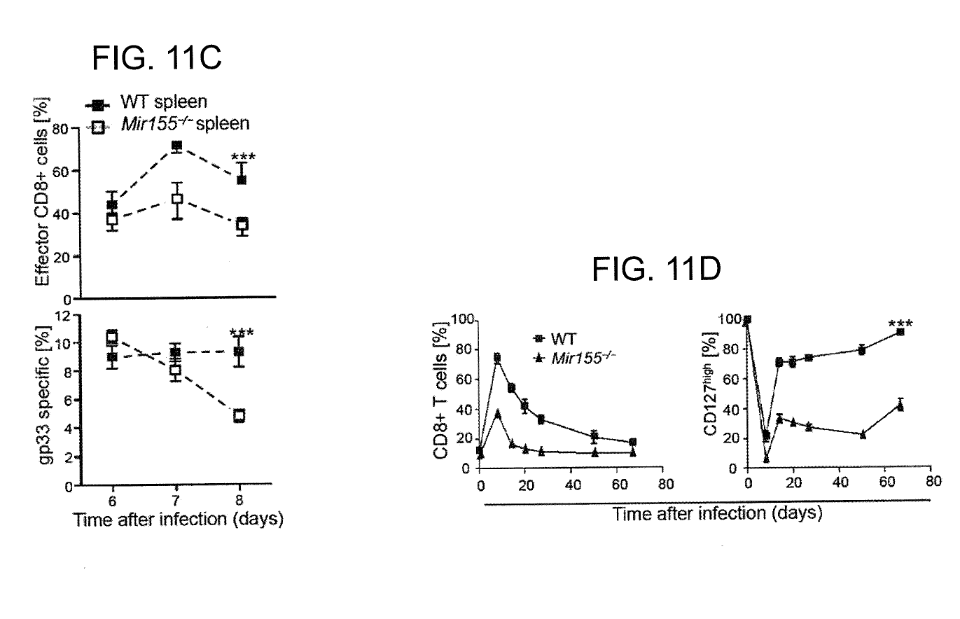

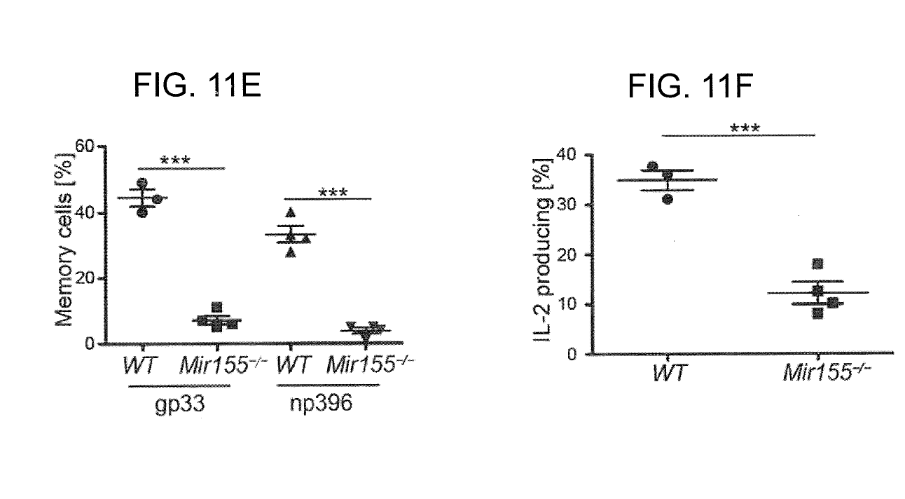

To determine the role of miR-155 in activated CD8⁺ T cells, the expansion of effector cells was monitored following acute LCMV WE strain infection in the presence or absence of miR-155. The percentage, number, and phenotype of naïve Mir155^{-/-} CD8⁺ T cells in blood and spleen did not differ from those in wild-type mice before infection. In contrast, both the percentage and number of total CD8⁺ T cells as well as virus gp33 tetramer specific CD8⁺ effector T cells were substantially reduced in spleen and blood of Mir 155^{-/-} mice at the peak of the response, as shown in FIGS. 11A and 11B. Following the expansion of CD44⁺ effector cells in the blood and spleen from days 6 to 8, impaired effector CD8⁺ cell accumulation was observed in spleen, liver, and blood of Mir155^{-/-} mice, as shown in FIG. 11C. Despite a defect in the magnitude of effector T cell responses, Mir155^{-/-} animals were capable of controlling viral replication and clearing the virus, as also confirmed by the lack of CD44 upregulation on adoptively transferred naïve LCMV specific P14 T cells. In addition, CD8⁺ T cells differentiated into phenotypically and functionally cytolytic effector cells similar to wild-type cells during LCMV infection. Circulating T cells in Mir155^{-/-} mice exhibited not only a defect in the expansion at the peak of the immune response but also a more rapid contraction compared to wild-type animals, as shown in FIG. 11D. Moreover, CD127⁺CD62L⁺KLRG1⁻ memory cells were strongly reduced in the gp33 and np396 tetramer⁺ CD8⁺ T cells in blood, liver, and spleen of Mir155^{-/-} mice three months after infection, as shown in FIG. 11E. Consistent with these findings, IL-2 production, a hallmark of central memory cells, was strongly diminished in Mir155^{-/-} mice after stimulation with gp33 peptide, as shown in FIG. 11F. In this immune memory context, a deficient CD4⁺ effector T cell activation also was observed on day 8 of the response in Mir155^{-/-} mice.

The results of this example demonstrate that miR-155 plays a role in inducing a robust T cell expansion, but not effector functions, as well as for a memory phenotype response upon an acute LCMV infection.

### EXAMPLE 9

This example demonstrates that intrinsic expression of miR-155 in CD8⁺ T cells promotes proliferation and limits apoptosis of effector CD8⁺ T cells.

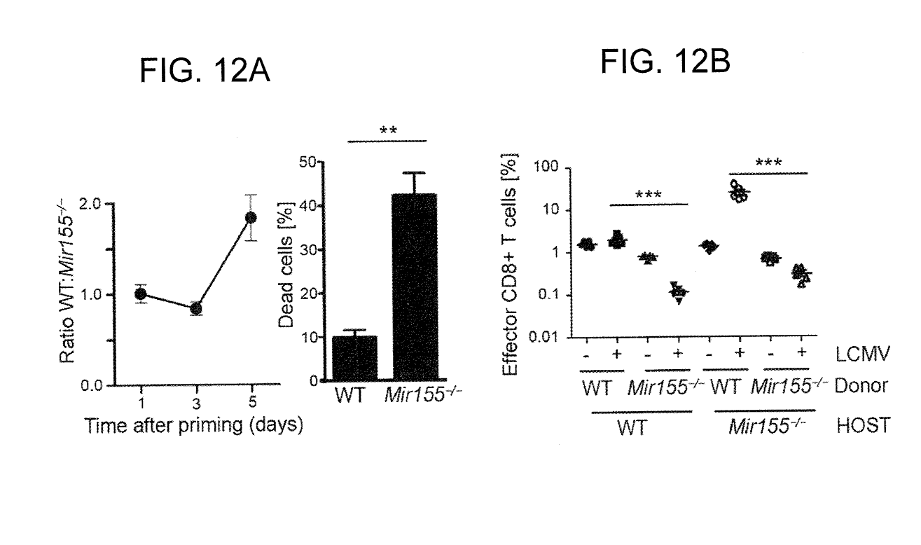

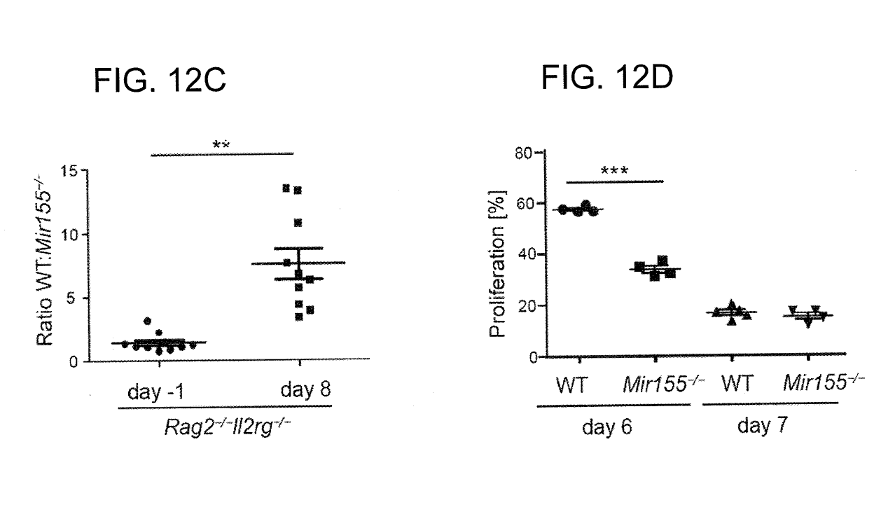

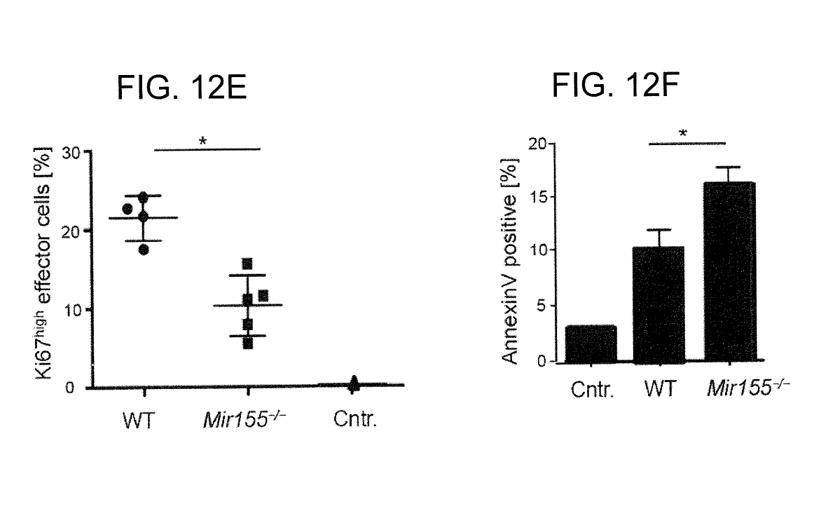

To investigate whether the defective expansion of CD8⁺ T cells was cell-intrinsic, naïve wild-type and congenic Mir155^{-/-} OT-1 CD8⁺ T cells were cocultured together with peptide pulsed dendritic cells and the OT-1 cell ratio was analyzed. After 5 days, wild-type CD8⁺ T cells outnumbered Mir155^{-/-} cells and the abundance of dead cells was strongly increased among Mir155^{-/-} T cells, as shown in FIG. 12A. To assess these parameters in vivo, equal numbers of congenic polyclonal wild-type and Mir155^{-/-} CD8⁺ T cells were co-transferred into either wild-type or Mir155^{-/-}hosts, which were then infected with LCMV. Despite the initial low frequency of wild-type CD8⁺ T cells transferred in miR-155 ablated hosts (i.e., about 1% of CD8⁺ T cells in blood before infection), these cells expanded to about 30% of the CD8⁺ T cells at the peak of the response. In contrast, the frequency of Mir155^{-/-} CD8⁺ T cells transferred into wild-type hosts decreased upon infection, as shown in FIG. 12B, demonstrating a stronger response of wild-type compared to Mir155^{-/-} CD8⁺ T cells. When Rag2 and common γ chain (γc)-deficient hosts were engrafted with a 1:1 mix of wild-type and Mir155^{-/-} splenocytes, both populations reached similar frequencies after two months, indicating comparable homeostatic expansion, as shown in FIG. 12C. However, following LCMV infection, wild-type T cells again showed an advantage in expansion over their Mir155^{-/-} counterparts. To determine the basis for the impaired accumulation of virus-specific CD8⁺ T cells in the absence of miR-155, LCMV infected wild-type and Mir155^{-/-} mice were pulsed with BrdU, and proliferation and apoptosis were measured four hours later. The proliferation of Mir155⁻CD44⁺ effector CD8⁺ T cells was decreased compared to wild-type cells six days after infection, as shown in FIG. 12D. Additionally, the frequency of proliferating Ki67⁺ cells within the CD44⁺CD62L⁻ effector CD8⁺ T cells was reduced in Mir155^{-/-} mice, as shown in FIG. 12E. Finally, an increased frequency of AnnexinV⁺ apoptotic cells was observed in Mir155^{-/-} CD8⁺ T cells compared to wild-type effector CD8⁺ T cells 7 days after infection, as shown in FIG. 12F.

The results of this example demonstrate a cell-intrinsic role of miR-155 in the proliferation and survival of effector CD8⁺ T cells in response to LCMV infection but not for homeostatic expansion in lymphopenic hosts.

### EXAMPLE 10

This example demonstrates that miR-155 is critical for effector CD8⁺ T cell accumulation and virus control in chronic LCMV infection.

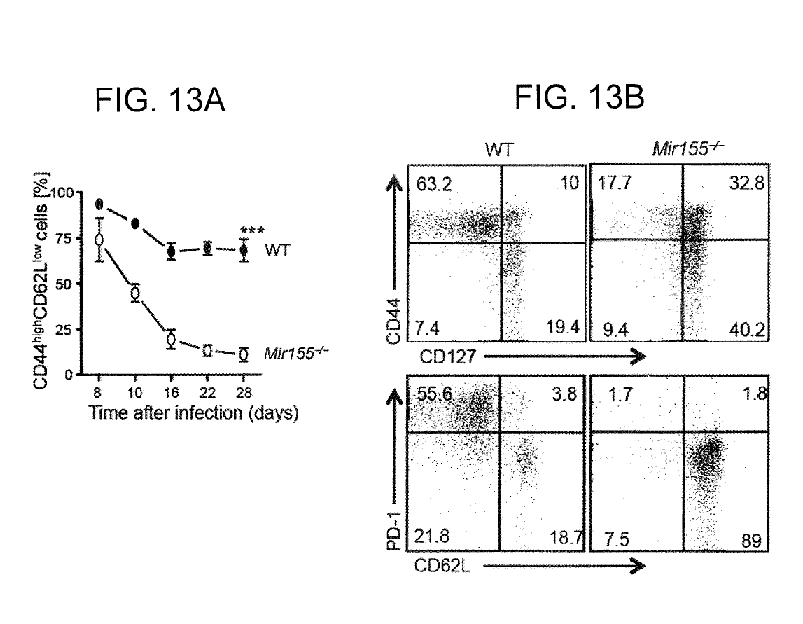

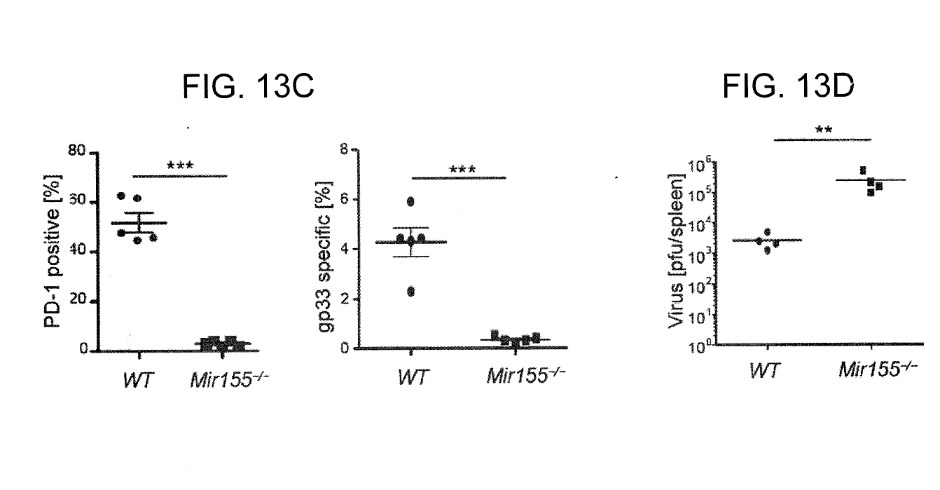

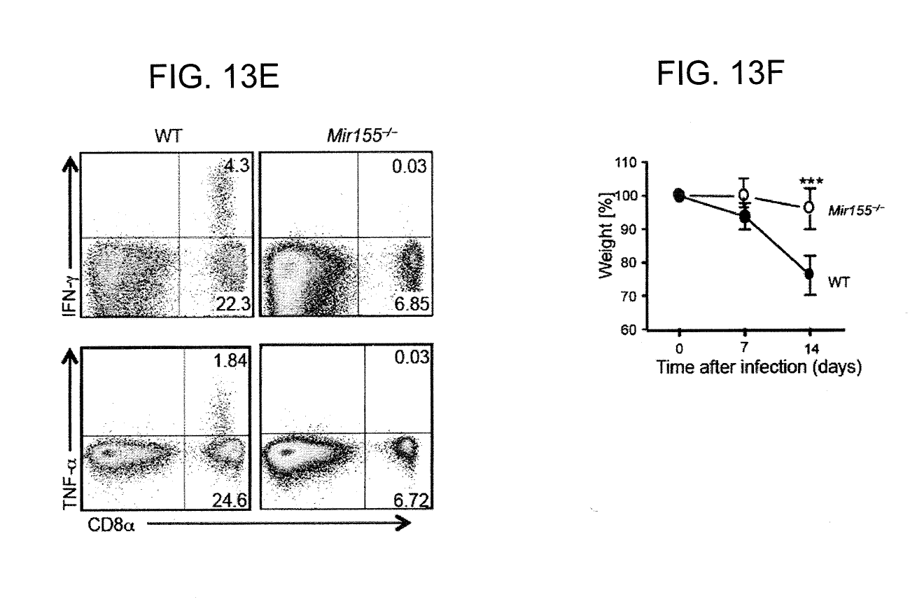

Based on the strong impairment of effector CD8⁺ T cell accumulation in low dose LCMV infection, the response of Mir155^{-/-} mice to high dose and long-lasting antigen exposure, which characterizes chronic infections and cancer, was determined. Mice were inoculated with 2×10⁶ pfu of LCMV clone 13, which caused a chronic infection for several weeks (Moskophidis et al., Nature, 362: 758-761 (1993); and Salvato et al., J. Virol., 65: 1863-1869 (1991)). While wild-type mice mounted a robust effector CD8⁺ T cell response with high percentages of CD44⁺CD62L⁻ effector cells that were maintained overtime, Mir155^{-/-} mice progressively lost effector CD8⁺ T cells, as shown in FIG. 13A. The remaining CD44⁺ cells in spleen showed high CD 127 and CD62L expression, reminiscent of a memory phenotype, as shown in FIG. 13B. Percentages and numbers of gp33 tetramer positive cells were also strongly decreased in deficient mice 5 weeks and 3 months post-infection, as shown in FIGS. 13B and 13C. At this time, cells capable of producing effector cytokines in response to a cocktail of LCMV peptides in miR-155 ablated mice could not be detected, confirming the loss of most virus specific Mir155^{-/-} CD8⁺ T cells, and ruling out TCR downregulation that may appear as tetramer negative T cells, as shown in FIG. 13E. While about 50% of wild-type cells remained positive for PD-1, associated with T cell exhaustion, PD-1 was barely detectable on Mir155^{-/-} CD8⁺ T cells five weeks upon infection, as shown in FIG. 13C. Virus titers were elevated five weeks and two months post-infection in miR-155 ablated mice, as shown in FIG. 13D. Finally, wild-type but not miR-155 ablated mice showed symptoms of immunopathology such as shivering, hunching, and weight loss, suggesting a lower inflammatory response in the absence of miR-155, as shown in FIG. 13F.

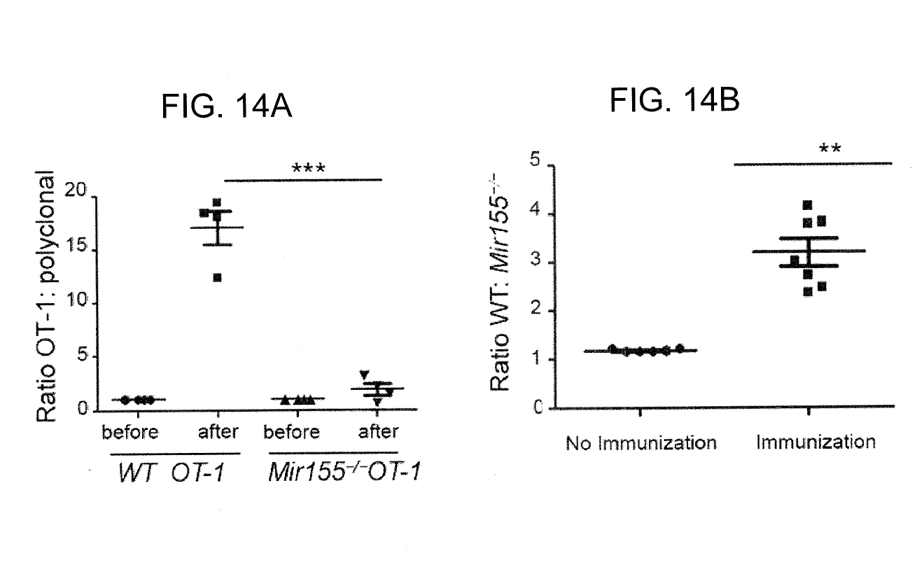

The role of miR-155 in CD8⁺ T cells in a clinically relevant vaccine setting characterized by limited adjuvant-induced inflammation also was determined. In this respect, polyclonal and OVA-specific OT-1 CD8⁺ T cells (either wild-type or Mir155^{-/-}) were cotransferred into wild-type mice before immunization with OVA peptide adjuvanted with IFA and CpG-ODNs. While the ratio of wild-type OT-1 to polyclonal cells strongly increased following immunization, there was only a minor increment in the ratio of Mir155^{-/-} OT-1 to polyclonal wild-type cells, as shown in FIG. 14A. Next, OT-1 cells from both wild-type and Mir155^{-/-}backgrounds were cotransferred into wild-type mice before immunization. Wild-type and Mir155^{-/-} cells were found in similar proportions, indicating a comparable survival after adoptive transfer. Following immunization, however, wild-type cells accumulated more efficiently than Mir155^{-/-} cells, as shown in FIG. 14B, whereas upregulation of CD44 and the proportion of cells producing IFN-γ were comparable.

The results of this example demonstrate that miR-155 plays a critical role in maintenance and survival of CD8⁺ effector T cells as well as virus control in chronic virus infections.

### EXAMPLE 11

This example demonstrates that targeting of SOCS-1 by miR-155 in effector CD8⁺ T cells enables cytokine responsiveness and accumulation.

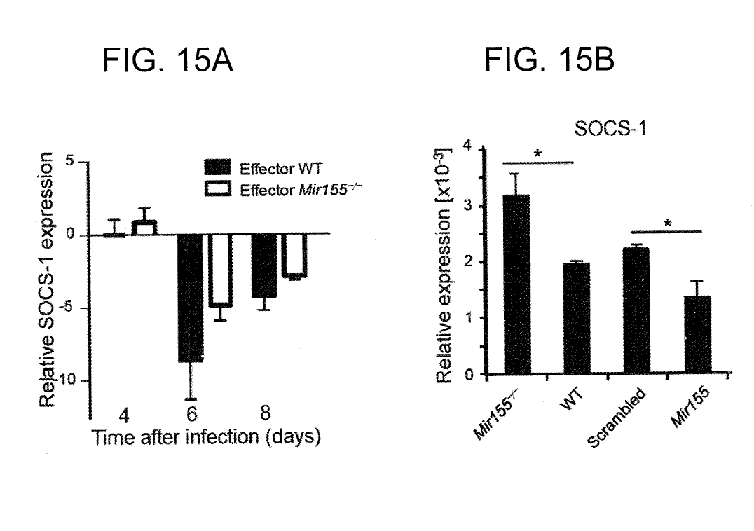

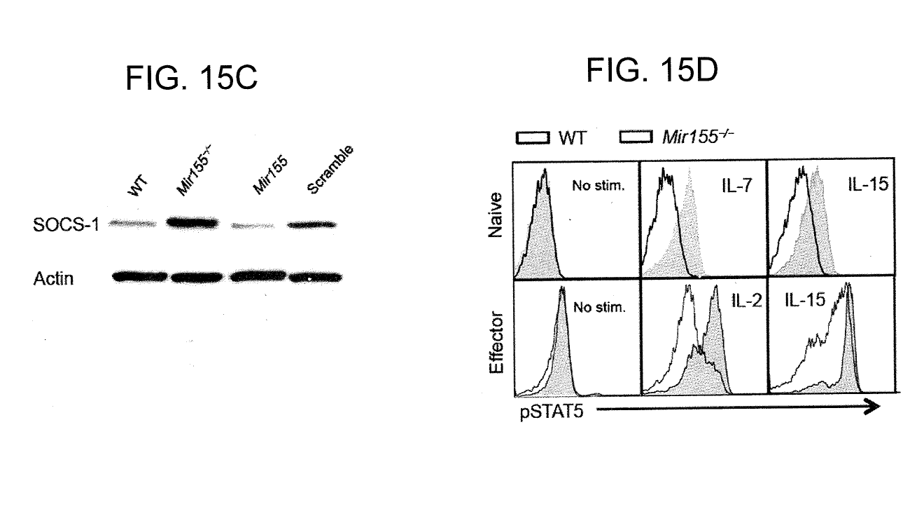

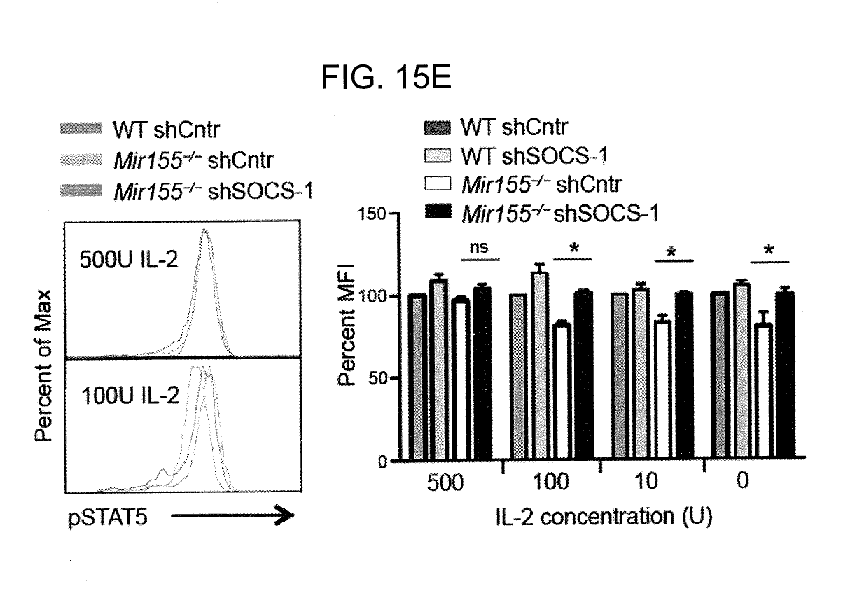

miR-155 has been shown to regulate γc-chain cytokine signaling by targeting SOCS-1 expression (D'Souza and Lefrancois, J. Immunol., 171: 5727-5735 (2003); Lu et al., Immunity, 30: 80-91 (2009); Wang et al., J. Immunol., 85: 6226-6233 (2010)). SOCS-1 regulation was assessed in splenic effector CD44⁺CD62L⁻CD8⁺ T cells during the response to acute LCMV infection of wild-type and Mir155^{-/-} mice. Both wild-type and Mir155^{-/-} CD8⁺T cells downregulated SOCS-1 on days 6 and 8 compared to CD62L⁺CD44⁻ naïve CD8⁺ T cells from non-infected mice, as shown in FIG. 15A. To more directly test if SOCS-1 was regulated by miR-155, SOCS-1 mRNA was measured in wild-type and Mir155^{-/-} CD8⁺ T cells as well as in cells overexpressing miR-155 or scrambled control miR. The amounts of SOCS-1 transcripts were inversely related to the cellular content of miR-155, with the highest concentration of SOCS-1 in Mir155^{-/-} cells and the lowest concentration of SOCS-1 in miR-155 transduced cells, as shown in FIG. 15B. These results were further confirmed at the protein level, indicating that miR-155 is a critical regulator of SOCS-1 translation in CD8⁺ T cells, as shown in FIG. 15C. To test whether the loss of miR-155 impaired γc chain cytokine signaling in CD8⁺ T cells by upregulating SOCS-1, STAT5 phosphorylation was compared in response to IL-2, IL-7, or IL-15 in wild-type and Mir155^{-/-} cells. Stimulation of naïve and effector CD8⁺ T cells isolated 8 days after LCMV infection resulted in a limited phosphorylation of STAT5 in miR-155 ablated cells, thereby demonstrating an impaired cytokine signaling, as shown in FIG. 15D. Diminished STAT5 phosphorylation was not due to differential expression of the cytokine receptor chains CD25, CD122, CD127 or CD 132.

To further investigate whether the impaired cytokine signaling was dependent on the higher SOCS-1 concentration in Mir155^{-/-} CD8⁺ T cells, wild-type and Mir155^{-/-} CD8⁺ T cells were transduced with control or shSOCS-1 lentivirus. Although in vitro activation of T cells diminished the impact of miR-155 on cytokine signaling, a rescue of pSTAT5 generation in shSOCS-1 transfected Mir155^{-/-} cells was consistently detected, as shown in FIG. 15E. Baseline pSTAT5 expression was already higher in wild-type than in Mir155^{-/-} cells without additional IL-2 stimulation. The difference between wild-type and Mir155^{-/-} cells was still apparent with intermediate IL-2 concentrations, but disappeared with high IL-2 concentrations, thereby demonstrating that saturating amounts of IL-2 overcome the miR-155 and SOCS-1 dependent inhibition of cytokine signaling, as shown for regulatory T cells (Lu et al., supra).

The results of this example demonstrate a dynamic and differentiation-dependent regulation of SOCS-1 during the response to LCMV and suggest that Mir155^{-/-} CD8⁺ T cells have impaired cytokine signaling due to increased SOCS-1 expression.

### EXAMPLE 12

This example demonstrates that SOCS-1 restrains CD8⁺ T cell responses to virus and cancer.

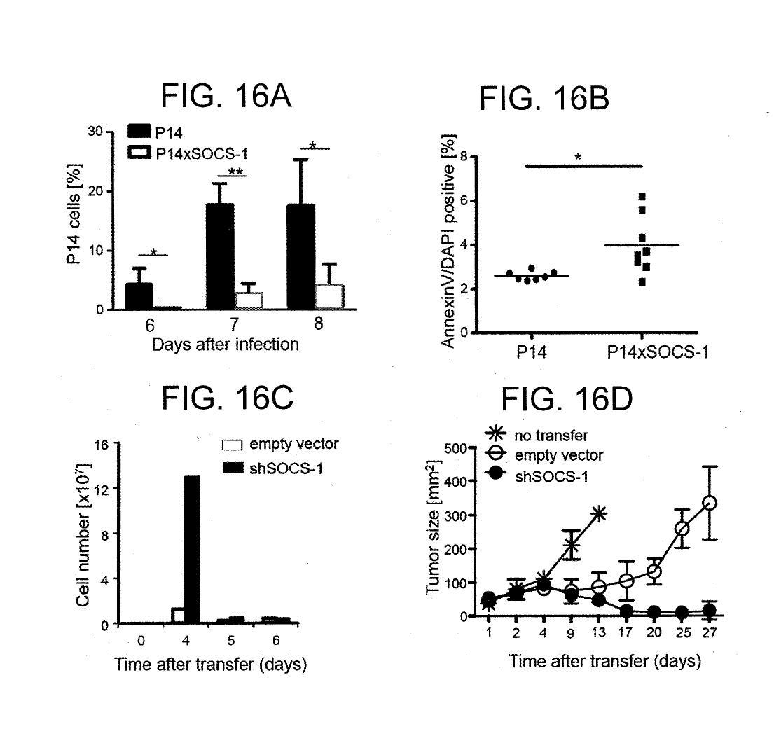

To test whether increased SOCS-1 expression recapitulated the impaired antigen-driven expansion of Mir155^{-/-} CD8⁺ T cells, SOCS-1 transgenic or wild-type P14 CD8⁺ T cells were adoptively transferred into congenic mice prior to infection with LCMV WE strain. The expansion of SOCS-1 transgenic P14 T cells in blood and spleen was reduced compared to P14 wild-type cells, as shown in FIG. 16A. Effector phenotype, granzyme B, and cytokine production were not impaired. However, enhanced apoptosis of SOCS-1 overexpressing cells was detected, as shown in FIG. 16B, thus phenocopying Mir155^{-/-} CD8⁺ T cells. To test if suppression of SOCS-1 could be therapeutically exploited to enhance the CD8⁺ T cell anti-tumor response, pmel-1 CD8⁺ T cells transduced with shSOCS-1 were adoptively transferred into tumor-bearing mice. SOCS-1 depletion by the construct was verified by immunoblot analysis. An increased expansion of cells expressing shSOCS-1 was detected in the spleen on day four compared to control, as shown in FIG. 16C, which was associated with profound tumor regression in mice that received shSOCS-1 transduced cells compared to untreated mice or mice treated with control cells, as shown in FIG. 16D.

The results of this example demonstrate that SOCS-1 negatively regulates the effector CD8⁺ T cell response to virus infection and cancer and emphasize the importance of SOCS-1 downregulation by miR-155 for efficient CD8⁺ T cell responses.

### EXAMPLE 13

This example describes experiments which further elucidate the signaling pathways affected by miR-155 overexpression in CD8⁺ T cells.

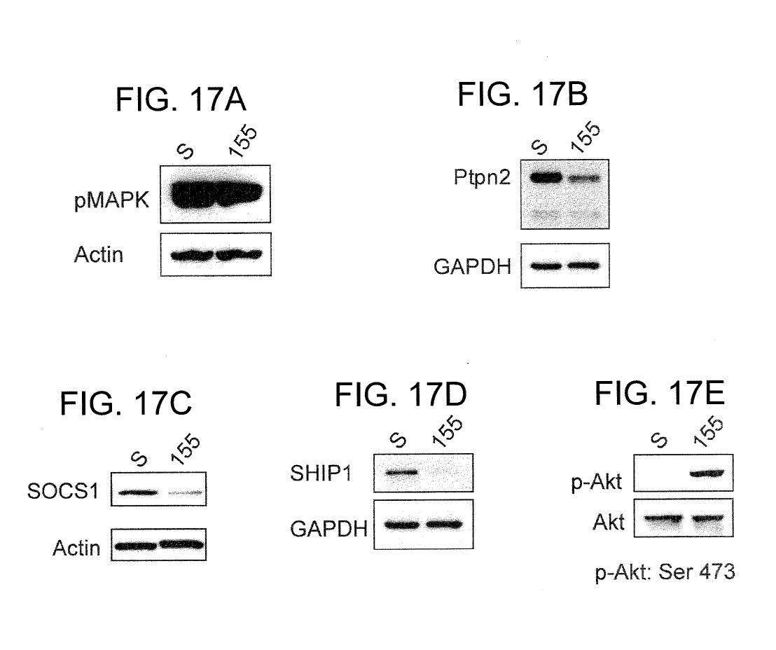

pmel-1 CD8⁺ T cells transduced with miR-155 or scrambled miR as described in Example 1 were assayed by immunoblot for expression of pMAPK, Ptpn2, SOCS1, SHIP1, and p-Akt. miR-155 overexpression in CD8⁺ T cells inhibited Ptpn2, SOCS1, and SHIP1 expression, as shown in FIGS. 17A-E.

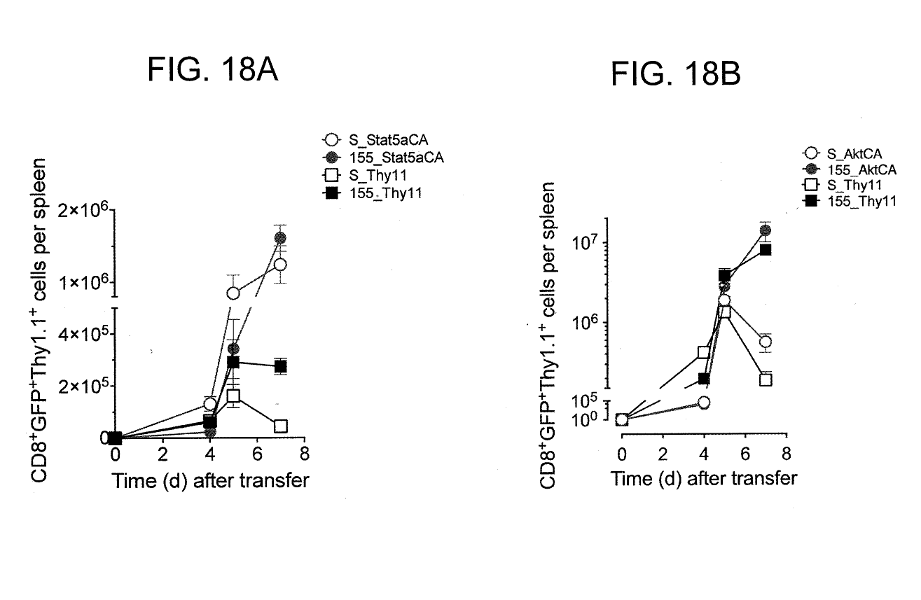

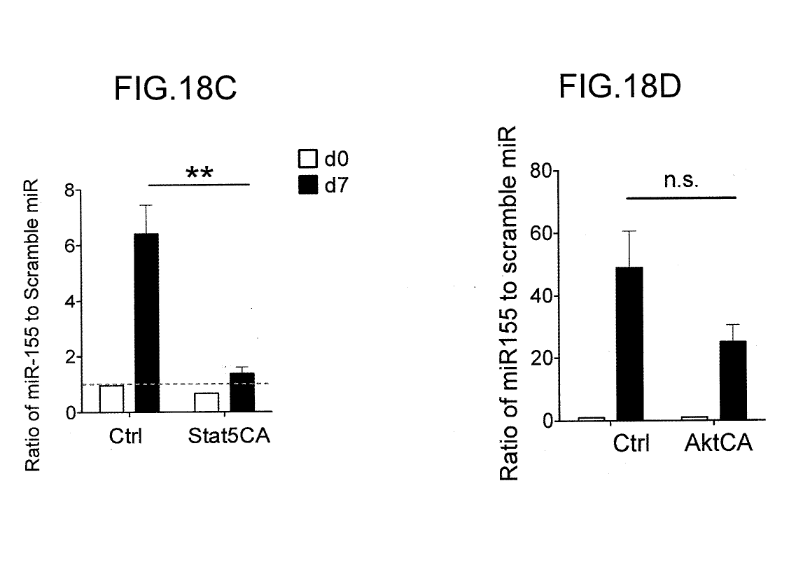

Separately, pmel-1 CD8⁺ T cells were transduced with a retrovirus encoding each of the following combinations of genes: Stat5CA (or AktCA)+scrambled miR; Stat5CA (or AktCA)+miR-155; Thy1.1+scrambled miR; and Thy1.1+miR-155. Four days after transduction, GFP⁺Thy1.1⁺ CD8⁺ T cells, which represent the co-expression of miR-155 and gene of interest, were sorted and adoptively transferred into C57BL/6 mice in conjunction with a recombinant retrovirus encoding gp-100. The number and cytokine releasing capacity of transferred cells were evaluated to assess the contribution of Stat5 and Akt, respectively, at specific time points after adoptive cell transfer. The expression of the constitutive Stat5a variant recapitulated the proliferative advantage conferred by miR-155 in a non-redundant manner, as shown in FIGS. 18A-D.

The results of this example demonstrate that miR-155-overexpressing T cells exhibit enhanced activity of Stat5 and Akt, and suggest that miR-155 acts through Stat5 rather than Akt in promoting CD8⁺ T cell expansion.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified CD8⁺ T cell comprising an antigen-specific T cell receptor (TCR) and an exogenous nucleic acid encoding a microRNA-155 (miR-155) molecule.

2. The isolated or purified CD8⁺ T cell of claim 1, wherein the TCR is specific for a cancer antigen.

3. The isolated or purified CD8⁺ T cell of claim 1, wherein the CD8⁺ T cell is a tumor infiltrating lymphocyte (TIL) or a peripheral blood lymphocyte (PBL) isolated from a host afflicted with cancer.

4. The isolated or purified CD8⁺ T cell of claim 1, wherein the CD8⁺ T cells is a human CD8⁺ T cell.

5. The isolated or purified CD8⁺ T cell of claim 1, wherein the exogenous nucleic acid encoding the miR-155 molecule is operably linked to a promoter.

6. The isolated or purified CD8⁺ T cell of claim 5, wherein the CD8⁺ T cell is transduced with a viral vector comprising the exogenous nucleic acid encoding the miR-155 molecule.

7. The isolated or purified CD8⁺ T cell of claim 5, wherein the CD8⁺ T cell is transfected with a plasmid comprising the exogenous nucleic acid encoding the miR-155 molecule.

8. The isolated or purified CD8⁺ T cell of claim 1, wherein the miR-155 is human miR-155, a precursor thereof, or an analog thereof.

9. The isolated or purified CD8⁺ T cell of claim 8, wherein the human miR-155 comprises the sequence of UUAAUGCUAAUCGUGAUAGGGGU (SEQ ID NO: 1).

10. The isolated or purified CD8⁺ T cell of claim 1, wherein the miR-155 is murine miR-155, a precursor thereof, or an analog thereof.

11. The isolated or purified CD8⁺ T cell of claim 10, wherein the murine miR-155 comprises the sequence of UUAAUGCUAAUUGUGAUAGGGGU (SEQ ID NO: 2).

12. A population of cells comprising at least one CD8⁺ T cell of claim 1.

13. A method of reducing the size of a tumor in a mammal, comprising administering to the mammal the population of cells of claim 12 in an amount effective to reduce the size of the tumor in the mammal.

14. The method of claim 13, wherein the cells of the population are autologous to the mammal.

15. The method of claim 13, wherein the method does not comprise administering to the mammal a treatment which is sufficient to cause depletion of immune cells.

16. The method of claim 13, wherein the method does not comprise administering to the mammal interleukin-2 (IL-2) or another cytokine which signals through the IL-2 gamma receptor.

17. The method of claim 13, wherein the method comprises vaccinating the mammal with one or more of (i) the antigen for which the TCR of the T cell is specific, (ii) an epitope of the antigen, and (iii) a vector encoding the antigen or the epitope.

18. The method of claim 13, wherein the method effectively treats cancer in the mammal.

19. A composition comprising at least one CD8⁺ T cell of claim 1, and a carrier therefor.

20. A method of reducing the size of a tumor in a mammal, comprising administering to the mammal the composition of claim 19 in an amount effective to reduce the size of the tumor in the mammal.
